SEQUENCE LISTING (15 sequences)

SEQ ID NO: 1 - protein, 386 aa, Gallus gallus
MGSIGAASMEFCFDVFKELKVHHANETIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTFVLMAMGITDVFSSSANLSGISSAESLKISQAVHAAHAEINEADREVVGSAEAGVDAASVSEEFRADHPFLFCIKHIATNAVLFFGRCVSP

SEQ ID NO: 2 - protein, 147 aa, Gallus gallus
MRSLLILVLCFLPLAALGKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 3 - protein, 705 aa, Gallus gallus
MKLILCTVLSLGIAAVCFAAPPKSVIRWCTISSPEEKKCNNLRDLTQQERISLTCVQKATYLDCIKAIANNEADAISLDGGQAFEAGLAPYKLKPIAAEVYEHTEGSTTSYYAVAVVKKGTEFTVNDLQGKTSCHTGLGRSAGWNIPIGTLLHRGAIEWEGIESGSVEQAVAKFFSASCVPGATIEQKLCRQCKGDPKTKCARNAPYSGYSGAFHCLKDGKGDVAFVKHTTVNENAPDQKDEYELLCLDGSRQPVDNYKTCNWARVAAHAVVARDDNKVEDIWSFLSKAQSDFGVDTKSDFHLFGPPGKKDPVLKDLLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPEKCVASSHEKYFGYTGALRCLVEKGDVAFIQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISSLKTCNPSDILQMCSFLEGK

SEQ ID NO: 4 - protein, 224 aa, Bos sp.
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 5 - protein, 190 aa, Bos sp.
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 6 - protein, 142 aa, Bos sp.
MMSFVSLLLVGILFHATQAEQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 7 - protein, 147 aa, Bos sp.
MKALVILGFLFLSVAVQGKVFERCELARTLKKLGLDGYKGVSLANWLCLTKWESSYNTKATNYNPSSESTDYGIFQINSKWWCNDGKTPNAVDGCHVSCRELMENDIAKAVACAKHIVSEQGITAWVAWKSHCRDHDVSSYVEGCTL

SEQ ID NO: 8 - protein, 708 aa, Bos sp.
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 9 - protein, 5 aa, Artificial Sequence
  Description of Artificial Sequence Synthetic peptide
GGGGS

SEQ ID NO: 10 - protein, 22 aa, Artificial Sequence
  Description of Artificial Sequence Synthetic peptide
MRIAMHLPLLLLYIFLLPLSGA

SEQ ID NO: 11 - protein, 20 aa, Artificial Sequence
  Description of Artificial Sequence Synthetic peptide
FKQSATIILALIVTILWFML

SEQ ID NO: 12 - protein, 14 aa, Artificial Sequence
  Description of Artificial Sequence Synthetic peptide
RGNTAKATYDLYTN

SEQ ID NO: 13 - protein, 386 aa, Columba livia
MASIGAASAEFSFEVFDELKAQYPNQNIIFAPLSILSALSMLYLGARGNTKAQIDKVVHFDKITGSGETVESQCSTSVNVHTSLKDTFIQITKPSDNYSLSFASRLYAEETFQILPEYIQCVKELYKESVETVSFQKAADQARELINSWAESQTNGMIRNILQPGSVDPQTEMVLVNAIYFKGVWAKAFKVEGTQTMPFRVTEQESKPVQMMYQLGSFKVTDLPPEKMRILELPYASGMLSMWVILPYDISGLEQIENAITFEKLQEWTSSNLMEEKTVKVYLPRMKMEEKYNLTSVLMGLGITDLFSSSADLSGISSVERLKVSEAVHQAFVEIDEAGSEAAGIAGAAVKDESASPEFKVDHPFLFLIRHNPTDGILFLARCVSP

SEQ ID NO: 14 - protein, 339 aa, Artificial Sequence
  Description of Artificial Sequence Synthetic polypeptide
MSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 15 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

(19) United States
(12) Patent Application Publication
(10) Pub. No.: US 2020/0337336 A1
(43) Pub. Date: Oct. 29, 2020

(54) RECOMBINANT YEAST AS ANIMAL FEED

(71) Applicant: Pando Nutrition Inc., Decatur, GA (US)

(72) Inventors: Craig Rouskey, Oakland, CA (US); Douglas Manofsky, Decatur, GA (US)

(21) Appl. No.: 16/962,998

(22) PCT Filed: Jan. 18, 2019
(86) PCT No.: PCT/US2019/014348

§ 371 Date: Jul. 17, 2020

Related U.S. Application Data
(60) Provisional application No. 62/619,053, filed on Jan. 18, 2018.

(51) Int. Cl. A23K 20/147; C12N 9/36; C07K 14/77; C07K 14/47; C07K 14/435; C07K 14/79; A23K 50/75

(52) U.S. Cl. CPC A23K 20/147; C12N 9/2462; C12Y 302/01017; A23K 50/75; C07K 14/4732; C07K 14/435; C07K 14/79; C07K 14/77

(57) ABSTRACT

Provided herein are transgenic direct fed microbial strains and direct feed compositions for feeding to animals; method of generating and using such compositions to as probiotic, to increase probiotics, and in egg-laying animals, to lower cholesterol in eggs and enhance yolk color.

### CROSS-REFERENCES TO RELATED APPLICATIONS

This application claims priority benefit of U.S. provisional application No. 62/619,053, filed Jan. 18, 2018, which application is herein incorporated by referenced for all purposes.

### BACKGROUND OF THE INVENTION

The commercial livestock industry is facing the demise of large-scale antibiotic use as growth promoters, as the negative impacts of global overuse of antibiotics as prophylactics leads to the generation and transfer of antibiotic-resistant pathogens to consumers. Probiotics, also known as Direct Fed Microbials, are poised to take center stage as a readily available, scalable and more sustainable solution to commercial animal health. As antibiotic use declines, the need for novel, non-antibiotic growth promoters, prophylactics and treatments for common gut-associated pathogens is emerging. To date, most direct fed microbials used in the poultry industry have been derived from autochthonous, non-transgenic strains. Autochthonous probiotic strains are microbes that are naturally occurring in the host microbiome; while allocthonous strains are not naturally present in the host microbiome.

Current methodologies in direct fed microbial administration involve pelleting of live microbial cells. The pelleting processes generates excessive heat and ultimately kills a significant fraction of the direct fed microbial strains, thus reducing the efficacy of some direct fed microbial supplements.

### BRIEF SUMMARY OF THE INVENTION

As described in the present disclosure, using recombinant DNA technology to engineer allochthonous probiotic strains of microbes can yield significant benefits to commercial livestock industries. These probiotic strains are both biologically effective, and extremely cost-efficient to manufacture, removing the need for expensive protein purification processes, and assuring that bioactive or nutritional proteins are efficiently delivered to the gut of livestock. Further, allochthonous direct fed microbials as described herein have advantages, as they do not naturally colonize the gut of the host livestock animal and naturally leave the host after treatment has concluded.

Further described herein are enrobing processes that allow for the application to and recovery of direct fed microbials from animal feed in vitro, and recoverable direct fed microbial strains from the microbiome of animals following consumption of enrobed feed, ex vivo.

In one aspect, the present disclosure thus provides compositions designed to provide maximal health benefits to livestock. Specifically, in some embodiments, the invention disclosed herein provides compositions that allow for the production of animal-protein fortified yeast serving as nutritional, functional, and/or broad-spectrum direct fed microbials for animal wellness. The modified strain producing the recombinant protein(s) is expected to have implications on animal health, microbiome development, and animal illness amelioration. The application of probiotics to animal feed via an enrobing process also facilitates scalable and efficient delivery of direct fed microbial strains to livestock.

In some embodiments, provided here are yeast or fungi host cells genetically modified to expresss an amount of a recombinant protein selected from the group consisting of: a recombinant alpha-lactalbumin, a recombinant ovalbumin, a recombinant lactoferrin, a recombinant lysozyme, a recombinant lactoperoxidase, a recombinant osteopontin, a recombinant haptocorrin, a recombinant alpha-amylase 1, a recombinant bile-salt stimulated lipase, a recombinant alpha-1-antitrypsin, a recombinant myeloperoxidase, a recombinant folate binding protein, a recombinant insulin-like growth factor 1 (IGF-1), a recombinant epidermal growth factor (EGF), a recombinant orosomucoid, a recombinant alpha-1-antichymotrypsin, a recombinant alpha-1-b-glycoprotein, a recombinant fetuin-A, a recombinant alpha-enolase, a recombinant alpha-S1-casein, a recombinant kappa casein, a recombinant beta-casein, a recombinant alpha-s2-casein, a recombinant caseinomacropeptide, a recombinant copine-5, a recombinant hapto-globin, a recombinant hemoglobin subunit delta, a recombinant lactadherin, a recombinant CD14, a recombinant mucin-1, a recombinant mucin-16, a recombinant mucin-4, a recombinant serum albumin, a recombinant serum transferrin, a recombinant tenascin, a recombinant thrombospondin-1, a recombinant transthyretin, a recombinant vitamin D-binding protein, a recombinant vitronectin protein, and a functional derivative of each protein, wherein each said protein has a glycosylation pattern associated with translation in a yeast, Aspergillus, or Bacillus cell, and wherein the composition is added to an animal feed via an enrobing process.

In some cases, the amount of recombinant protein in the transgenic direct fed microbial strain is between 5×10⁻¹⁵ g and 5×10⁻¹² g, or 0.1% to 100% of total cellular protein weight per cell. In some instances, the composition is a supplement of a secondary animal feed, applied to said feed via an enrobing process.

In some cases, the recombinant protein is secreted from the direct fed microbial strain, is anchored into the membrane of the direct fed microbial strain, or is intracellularly expressed within the direct fed microbial strain allowing for efficient delivery of nutritional and bioactive proteins to the gut of livestock.

In some embodiments, recombinant alpha-lactalbumin is expressed from a vector within a microbe encoding an avian, murine, a leporine, a canine, a feline, a porcine, a bovine, an ovine, a caprine, or an equine protein. In some instances, the microbe is of the Saccharomyces genus. In some instances, the yeast is S. cerevisiae. In some instances of this invention, the Aspergillus is Aspergillus oryzae, Aspergillus niger, or Aspergillus nidulans. In some instances, the Bacillus is Bacillus megaterium or Bacillus subtilis.

In some aspects, this invention relates to a composition comprising a recombinant yeast from the Saccharomyces genus, wherein said recombinant yeast comprises at least one heterologous alpha-lactalbumin, ovalbumin, lactoferrin, lysozyme, lactoperoxidase, osteopontin, haptocorrin, alpha-amylase 1, bile-salt stimulated lipase, alpha-1-antitrypsin, myeloperoxidase, folate binding protein, insulin-like growth factor 1 (IGF-1), epidermal growth factor (EGF), orosomucoid, alpha-1-antichymotrypsin, alpha-1-b-glycoprotein, fetuin-A, alpha-enolase, alpha-S1-casein, kappa casein, beta-casein, alpha-s2-casein, caseinomacropeptide, copine-5, hapto-globin, hemoglobin subunit delta, lactadherin, CD14, mucin-1, mucin-16, mucin-4, serum albumin, serum transferrin, tenascin, thrombospondin-1, transthyretin, vitamin D-binding protein or vitronectin generating heterologous nucleic acid sequence. In some aspects, the recombinant yeast comprises heterologous nucleic acid sequences that encode an avian, murine, a leporine, a canine, a feline, a porcine, a bovine, an ovine, a caprine, or an equine protein.

In some cases, an animal feed may comprise one or more of the recombinant direct fed microbial strains described above. In other aspects, this invention discloses supplements to an animal food comprising one or more of the previously described recombinant strains. In some aspects, the recombinant strain expresses a recombinant alpha-lactalbumin. In some aspects, the recombinant protein is a recombinant ovalbumin. In some aspects, the recombinant protein is a recombinant lactoferrin. In some aspects, the recombinant protein is a recombinant lysozyme. In some aspects, the recombinant protein is a recombinant lactoperoxidase. In some aspects, the recombinant protein is a recombinant osteopontin. In some aspects, the recombinant protein is a recombinant haptocorrin. In some aspects, the recombinant protein is a recombinant alpha-amylase 1. In some aspects, the recombinant protein is a recombinant bile-salt stimulated lipase. In some aspects, the recombinant protein is a recombinant alpha-1-antitrypsin. In some aspects, the recombinant protein is a recombinant myeloperoxidase. In some aspects, the recombinant protein is a recombinant folate binding protein. In some aspects, the recombinant protein is a recombinant insulin-like growth factor 1 (IGF-1). In some aspects, the recombinant protein is a recombinant epidermal growth factor (EGF). In some aspects, the recombinant protein is a recombinant orosomucoid. In some aspects, the recombinant protein is a recombinant alpha-1-antichymotrypsin. In some aspects, the recombinant protein is a recombinant alpha-1-b-glycoprotein. In some aspects, the recombinant protein is a recombinant fetuin-A. In some aspects, the recombinant protein is a recombinant alpha-enolase. In some aspects, the recombinant protein is a recombinant alpha-S1-casein. In some aspects, the recombinant protein is a recombinant kappa casein. In some aspects, the recombinant protein is a recombinant beta-casein. In some aspects, the recombinant protein is a recombinant alpha-s2-casein. In some aspects, the recombinant protein is a recombinant caseinomacropeptide. In some aspects, the recombinant protein is a recombinant copine-5. In some aspects, the recombinant protein is a recombinant hapto-globin. In some aspects, the recombinant protein is a recombinant hemoglobin subunit delta. In some aspects, the recombinant protein is a recombinant lactadherin. In some aspects, the recombinant protein is a recombinant CD14. In some aspects, the recombinant protein is a recombinant mucin-1. In some aspects, the recombinant protein is a recombinant mucin-16. In some aspects, the recombinant protein is a recombinant mucin-4. In some aspects, the recombinant protein is a recombinant serum albumin. In some aspects, the recombinant protein is a recombinant serum transferrin. In some aspects, the recombinant protein is a recombinant tenascin. In some aspects, the recombinant protein is a recombinant thrombospondin-1. In some aspects, the recombinant protein is a recombinant transthyretin. In some aspects, the recombinant protein is a recombinant vitamin D-binding protein. In some aspects, the recombinant protein is a recombinant vitronectin protein.

In some aspects, this invention relates to a method for feeding an animal, comprising an animal feed comprising from about 0.01% to 100% of a direct fed microbial composition described above, wherein the amounts are by total weight of the food, and providing the animal feed to the animal for ingestion via an enrobing process.

In a further aspect, provided here in is a method for feeding an animal in which the transgenic direct fed microbial is applied to an animal feed pellet via an enrobing process. The enrobing agent consists of direct fed microbial cells from about 0.001% to about 99.99% of the composition; water from about 0.01% to 99.99% of the composition; and oils: Peanut Oil, Soybean Oil, Canola Oil, Coconut Oil, Corn Oil, Olive Oil, Extra Virgin Olive Oil, Sesame Oil, Fish Oil, Vegetable Oil, Avocado Oil, Pumpkin Seed Oil, Walnut Oil, Grapeseed Oil, Hemp Seed Oil, Flaxseed Oil, Palm Oil, and/or Sunflower Seed Oil from about 0.01% to 99.99% of the composition; and prebiotic yeast cell wall, mannanoligosaccharides (MOS), fructooligosaccharides (FOS), oligosaccharides, soluble and insoluble dietary fibers, beta-glucan, mannose, carrot powder, beet powder, red bell pepper powder, from about 0.01% to 99.99% of the composition, wherein the amounts are by total percent of the final feed.

The invention relates to a method of applying the enrobing agent to an animal feed pellet in which the enrobing agent is applied to the exterior of the feed pellet after pelleting, through a spray device and/or is directly added to the exterior of the feed pellets in an industrial mixer.

Additional illustrative aspects of the invention include, but are not limited to, the following. In one aspect, provided here is an animal feed supplement comprising a transgenic direct fed microbial strain is genetically modified to express at least one polypeptide as follows: an intracellularly expressed polypeptide selected from ovalbumin, bovine alpha-lactalbumin, bovine beta-casein, and bovine kappa casein, wherein the transgenic strain of has an altered amino acid profile compared to a counterpart control of the same strain that does not have the genetic modification; a membrane-anchored polypeptide selected from chicken lysozyme, bovine lysozyme, and chicken ovotransferrin; or a secreted polypeptide selected from chicken lysozyme, bovine lysozyme and ovotransferrin. In some embodiments, the at least one intracellularly expressed polypeptide, membrane-anchored polypeptide, or secreted polypeptide is encoded by a gene codon-optimized for expression in the microbial strain. In some embodiments, the transgenic direct fed microbial strain is a Saccharomyces cerevisiae strain.

In some embodiments, the transgenic direct fed microbial strain is genetically modified to express membrane-anchored chicken ovotransferrin. In other embodiments, the transgenic direct fed microbial strain is genetically modified to express membrane-anchored chicken or bovine lysozyme.

In some embodiments, the transgenic direct fed microbial strain is genetically modified to express at least one intracellularly expressed polypeptide selected from ovalbumin, bovine alpha-lactalbumin, bovine beta-casein, and bovine kappa casein, and the transgenic strain has an altered amino acid profile compared to a counterpart control of the same strain that does not have the genetic modification. In some embodiments, such an animal feed supplement increases fat adsorption in the animal to which it is feed. In some embodiments, the transgenic direct fed microbial strain is genetically modified to express an ovalbumin that lacks a secretion signal.

In some embodiments, the transgenic direct fed microbial strain is genetically modified to express secreted bovine or chicken lysozyme. In some embodiments, the transgenic direct fed microbial strain is genetically modified to express secreted ovotransferrin. In some embodiments, the transgenic direct fed microbial strain is a Saccharomyces cerevisiae genetically modified to express the at least one secreted polypeptide and the gene encoding the secreted polypeptide encodes the region of the polypeptide that is secreted fused to a yeast FAKS secretion signal.

In some embodiments, an animal feed supplement as described herein, e.g., in the preceding paragraphs, comprises a transgenic direct fed yeast strain, wherein the parent strain that is genetically modified to produce the transgenic direct fed strain is an allochthonous yeast species.

In some embodiments, the gene introduced into the direct fed microbial strain encoding the at least one expressed polypeptide is maintained in the transgenic direct fed microbial strain as an autonomously replicating vector. In other embodiments, the gene encoding the at least one expressed polypeptide is integrated into the genome transgenic direct fed microbial strain.

In a further aspect, provided herein is a method of formulating an animal feed comprising an animal feed supplement as described herein, e.g., in the preceding paragraphs, for feeding to animals, comprising enrobing the transgenic direct fed microbial strain, wherein the step of enrobing comprises re-suspending the transgenic strain in an emulsion of fat, water and prebiotics, and coating onto feed pellets. In some embodiments, the transgenic direct fed microbial strain is a Saccharomyces cerevisiae strain. Accordingly, in a further aspect, provided herein is an animal feed formulated by the method.

In a further aspect, provided herein is a method of increasing fat adsorption in an animal, where the method comprises feeding an animal a feed comprising a transgenic direct fed microbial strain genetically modified to express at least one intracellularly expressed polypeptide selected from ovalbumin, bovine alpha-lactalbumin, bovine beta-casein, and bovine kappa casein, expression of which alters the amino acid profile compared to a counterpart control of the same strain that does not have the genetic modification. In some embodiments the animal feed is formulated as described herein, e.g., in the preceding paragraph. In some embodiments, the transgenic direct fed microbial strain is a Saccharomyces cerevisiae strain. In some embodiments, the animal is a chicken.

In a further aspect, provided herein is a method of altering the content of gram positive bacteria in the microbiome of an animal, where the method comprises feeding an animal a feed comprising a transgenic direct fed microbial strain genetically modified to express secreted bovine or chicken lysozyme. In some embodiments the animal feed is formulated as described herein, e.g., in the paragraphs above. In some embodiments, the transgenic direct fed microbial strain is a Saccharomyces cerevisiae strain. In some embodiments, the animal is a chicken.

In a further aspect, provided herein is a method of altering the content of gram negative bacteria in the microbiome of an animal, where the method comprises feeding an animal a feed comprising a transgenic direct fed microbial strain genetically modified to express wherein the transgenic direct fed microbial strain expresses secreted ovotransferrin. In some embodiments the animal feed is formulated as described herein, e.g., in the paragraphs above. In some embodiments, the transgenic direct fed microbial strain is a Saccharomyces cerevisiae strain. In some embodiments, the animal is a chicken.

Also provided herein is an animal feed supplement comprising a transgenic direct fed microbial strain that is genetically modified to express a heterologous recombinant protein, or fragment thereof, selected from the group consisting of: an alpha-lactalbumin, an ovalbumin, a lactoferrin, a lysozyme, a lactoperoxidase, an osteopontin, a haptocorrin, an alpha-amylase 1, a bile-salt stimulated lipase, an alpha-1-antitrypsin, a myeloperoxidase, a folate binding protein, an insulin-like growth factor 1 (IGF-1), an epidermal growth factor (EGF), an orosomucoid, an alpha-1-antichymotrypsin, an alpha-1-b-glycoprotein, a fetuin-A, an alpha-enolase, an alpha-S1-casein, a kappa casein, a beta-casein, an alpha-s2-casein, a caseinomacropeptide, a rcopine-5, a hapto-globin, a hemoglobin subunit delta, a lactadherin, a CD14, a mucin-1, a mucin-16, a recombinant mucin-4, a serum albumin, a t serum transferrin, a tenascin, a thrombospondin-1, a transthyretin, a vitamin D-binding protein, and a vitronectin protein. In some embodiments, the protein is expressed from an avian, murine, leporine, canine, feline, porcine, bovine, ovine, caprine, or equine gene that encodes the heterologous recombinant protein. In some embodiments, the protein is present in the transgenic direct fed microbial strain in an amount between 5×10⁻¹⁵ g and 5×10⁻¹² g, or 0.1% to 100% of total cellular protein weight. In some embodiments, the amino acid profile of the transgenic direct fed microbial strain is altered compared to a counterpart of the same strain that does not have the genetic modification. In some embodiments, the transgenic direct fed microbial strain is a yeast strain, e.g., of the Saccharomyces genus, such S. cerevisiae. Alternatively, the transgenic direct fed microbial strain may be of the genus Aspergillus, e.g., Aspergillus oryzae, Aspergillus niger, or Aspergillus nigulans. In other embodiments, the transgenic direct fed microbial strain may be of the genus Bacillus, e.g., Bacillus megaterium or Bacillus subtilis. In some embodiments, the Bacillus strain that is genetically modified to produce the transgenic direct fed microbial strain is autochthonous.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modification in various obvious respects, all without departing from the disclosure. Accordingly, the drawings and descriptions are to be regarded as illustrative in nature, and not as restrictive.

### BRIEF DESCRIPTION OF THE DRAWINGS

FIG. 1. Edited Amino Acid Sequences of Recombinant ovalbumin protein used in the creation of PN012-3, a recombinant strain of S. cerevisiae that expresses bovine alpha-lactalbumin, bovine kappa-casein, bovine beta-casein, and ovalbumin. Amino acid sequence 1 is ovalbumin from Rock Dove; Amino acid sequence 2 is a truncated ovalbumin as described herein. Amino acid sequence 3 is native hen ovalbumin sequence. Deletion of the first 49 amino acids allows Ovalbumin to remain inside the yeast cell.

FIG. 2. Plasmids used in the construction of an InvSc1 (PN002; S. cerevisiae)-based probiotic strain, PN012-3, expressing four key proteins that enhance nutrition. PN002 was chemically transformed with four plasmids encoding bovine alpha-lactalbumin, bovine beta-casein, bovine kappa-casein and truncated, hen ovalbumin.

FIG. 3. Protein Expression of PN012 clones following transformation into PN002. Cell lysates were analyzed for intracellular expression of 4 recombinant proteins by Western Analysis. Data indicate that all four proteins were properly expressed in PN012 strains.

FIG. 4. Expression of recombinant bovine and hen proteins alters the amino acid composition of yeast, making it a more nutritious probiotic. Briefly, strains, PN012-1, -3 and -7 were scaled and analyzed for their ability to alter the amino acid profiles of yeast. The amino acid analysis protocol uses chemical lysis to break down cellular protein into single amino acids that are analyzed via chromatography. The percentages of dry cellular weight are then calculated from the pMolar amounts of each amino acid. Data indicate that expression of four recombinant proteins resulted in an increase in key amino acids necessary for livestock development, PN012-3 showing the greatest improvement in these amino acids, collectively.

FIG. 5. Recombinant, probiotic yeast bind fat better than Whey Protein Concentrate and Enyzmatic Yeast Hydrolysate. Yeast cells were gently lysed to preserve the proteins in solution, quantitiated and bound to fat in an emulsion. The emulsion as then centrifuged and resulting micelles removed from solution. The protein remaining in solution was quantified by Bradford Assay and percent of bound protein calculated. Strain PN012-3, expressing four recombinant proteins, showed the greatest ability to bind find when compared to its counterparts.

FIG. 6. Plasmid insertion cassette and complete plasmid representation used in the construction of PN031—a recombinant PN002 expressing membrane-bound bovine lysozyme. The transmembrane signals, G4S linker, and GPI anchor domains are annotated.

FIG. 7. Expression of native bovine lysozyme (PN031) and mutated bovine lysozyme (A114P, PN024) on the surface of cells transformed with the plasmids indicated in FIG. 6. Mutated clones are in windows 8, 9, and 10. Wild Type lysozyme is depicted in windows 11, 12, 13, and 14. Clones 12 and 14 were selected for further use. Data indicate that both mutated bovine lysozyme, and the wild-type, native bovine lysozyme were both expressed on the surface of PN024, and PN031, respectively.

FIG. 8a. Knockout Cassette for the Homothalism Gene. The KANMX cassette was synthesized with 5′ and 3′ homologies to the BY4741 haploid yeast strain. The cassette was amplified by PCR, purified, and 4 ug were electroporated into BY4741. The recombination reaction was plated on 500 ug/mL G418 and resultant colonies screened for insertion.

FIG. 8b. Amplification of recombinant cells derived from KanMX insertion into the homothallism gene. Lane 1-Ladder, 2-PN077-1, 3-PN077-2, 4-PN077-3, 5-PN077-4, 6-PN077-5, 7-PCR Negative Control, 8-BY4741 non-transformed, 9-Positive control (cassette). PN077-4 was chosen for the following steps in which the Knock-in cassette (FIG. 9) was transformed into the KanMX locus.

FIG. 9. Knock-in Cassette designed for the Homothalism locus. Homologies to the homothallism gene flanked the Tef promoter, FAKS signal sequence, the codon optimized, native chicken Lysozyme gene, and Tef terminator. The homologies allow for insertion of the cassette into the Homothallism locus within BY4741. Subsequent transformants were screened for G418 sensitivity, and their ability to produce lysozyme via Zone of Inhibition Assay. The resulting strains were termed PN078.

FIG. 10a. Zone of Inhibition indicating a functioning, bioactive lysozyme secreted via the FAKS signal sequence from strains BY4741 and PN002 (InvSc1). Strains PN066-11 (left) and PN067-31 (right) were considered positive. Strain PN066-11 is PN002 with the native, hen lysozyme gene inserted into a plasmid (FIG. 11) that is then transformed into PN002. PN067-31 is BY4741 transformed with a chicken lysozyme construct depicted in FIG. 11. Strains PN077-4-1 through -16 are stable integrants expressing lysozyme from the integration cassette pictured in FIG. 9. Lysozyme is toxic to M. luteus, so when the strains expressing the secreted protein are plated along with M. luteus, the lysozyme prevents the growth of the bacteria in a zone of inhibition. The zone of inhibition is dependent on the amount of protein being secreted by the strain.

FIG. 10b. Kill assay of PN066-11. Supernatant from PN066-11 was added (100 uL) to a culture of Micrococcus luteus in 1× Phosphate Buffered Saline. The cultures were allowed to incubate for 24 hours at room temperature and assessed for lysis by Optical Density (OD) at 600 nm. The positive control and PN066-11 supernatants killed Micrococcus luteus at 24 hours post incubation. The negative control did not.

FIG. 11. Hen Lysozyme and Ovotransferrin Plasmids used to transform BY4741 and InvSc1 (PN002). The figure depicts the insertion site of the lysozyme or ovotransferrin genes into pD1214-FAKS plasmid backbone. The resultant plasmids were propagated in E. coli, purified, sequenced, and chemically transformed into PN002 or BY4741.

FIG. 12a. In vitro recovery of yeast from chicken feed following enrobing process.

FIG. 12b. In vitro recovery of recombinant yeast after enrobing process

FIG. 13a. Effective prebiotic delivery was assessed by yolk color change.

FIG. 13b. Direct fed microbial strains were effectively delivered and are present at day 28. Microbiome data indicate an absence of Saccharomyces cerevisiae on days 0 (101418) and a presence at day 28 (11112018).

FIG. 13c. Data indicate a decrease in Escherichia species and an increase of Lactobacillus species associated with the prebiotic-probiotic enrobed supplement. Samples taken at day 0 (indicated by “10142018”) contained Escherichia species, while samples taken from the same flocks at day 28 (indicated by “2018_11_11”) contained no Escherichia and an increase of Lactobacillus species, which was an anticipated effect of yeast direct fed microbial administration.

### DETAILED DESCRIPTION

The present disclosure is related to the production of recombinant direct fed microbials and compositions for animal feed additives, and methods of providing such compositions to animals. For instance, the product of methods and compositions of the present disclosure can be fortified yeast, fungi, or Bacillus species servings used as an additive to animal feed, animal feed additive, animal water, or the product can be provided as an independent additive. Elements of the present disclosure are related to, without limitation, strain engineering, specific expression levels of the recombinant protein secreted from the yeast cell, the construction of recombinatory shuttle vectors or integration cassettes.

The present disclosure provides a method of improving the gut health of backyard, small-scale industrial, and commercial livestock.

### Terminology

A “microbial,” host cell, as used herein, generally refers to a bacterium, yeast or fungus.

A “direct fed microbial,” as used herein, generally refers to a live bacterium, yeast, or fungus used as a probiotic for backyard, small-scale, or commercial livestock. A “direct fed microbial” is an essentially pure, living strain or a multi-strain, living composition intended for or suitable for being added to food or feed. Direct fed microbials are able to convert traditionally non-viable nitrogen and carbon into elements that are ultimately usable by the host. In particular it is a substance that by its intended use is becoming a component of a food or feed product or affects any characteristics of a food or feed product. Thus, for example, a lysozyme-expressing direct fed microbial is understood to refer to a living recombinant cell that produces lysozyme which is not a natural constituent of the main feed or food substances or is not present at its natural concentration therein, e.g., the lysozyme expressing strain is added to the feed separately from the feed substances, alone or in combination with other feed additives.

In some embodiments, the direct fed microbial is “allochthonous,” which as used herein, refers to a direct fed microbial strain that is not native to the host microbiome. The advantage of using an allochthonous direct fed microbial strain is that it does not colonize the gut beyond periods of administration and will not outcompete the native microbiome. While there are regulatory advantages to using this type of direct fed microbial strain (e.g. the organism will not persistently eliminate all gram-negative bacteria), the primary advantage is in controlling the administration of the direct fed microbial strain. Conversely, “autochthonous,” as used herein refers to a direct fed microbial strain that persists in the microbiome of the host following administration. Autochthonous direct fed microbials deliver a constant stream of benefits to the host and have the capability of colonizing the gut well beyond administration period.

“Altered levels,” as used herein, generally refers to the level of expression in transformed or transgenic cells or organisms that differs from a reference level or profile, such as the level of expression of normal or untransformed cells or organisms.

“Expression,” as used herein, generally refers to the transcription and/or translation of an endogenous or heterologous gene in a host cell. For example, in the case of antisense constructs, expression may refer to the transcription of the antisense DNA only. In addition, expression refers to the transcription and stable accumulation of sense (mRNA) or functional RNA. Expression may also refer to the production of protein.

“Recombinant” or “transgenic,” as used herein refers to a strain of bacterium, fungus, or yeast that expresses heterologous proteins following genetic manipulation.

“Strain” or “Strains,” as used herein, generally refers to a recombinant or transgenic organism that is either bacterium, fungi, or yeast. The “parental strain” is the organism from which the recombinant or transgenic strains are derived.

“Expression cassette,” as used herein, generally means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to a particular external stimulus, e.g. biotic or abiotic stress, or external chemical stimuli.

The term “gene,” as used herein generally refers to any segment of nucleic acid associated with a biological function. Thus, genes include coding sequences and/or the regulatory sequences required for their expression. For example, gene refers to a nucleic acid fragment that expresses mRNA, or a specific protein, including regulatory sequences. Genes also include non-expressed DNA segments that, for example, form recognition sequences for other proteins. Genes can be obtained from a variety of sources, including cloning from a source of interest or synthesizing from known or predicted sequence information, and may include sequences designed to have desired parameter.

The term “polynucleotide” or “nucleic acid,” as used herein, generally refers to a polymeric form of nucleotides of any length, either ribonucleotides or deoxyribonucleotides, that comprise purine and pyrimidine bases, purines and pyrimidine analogues, chemically or biochemically modified, natural or non-natural, or derivatized nucleotide bases. Polynucleotides include sequences of deoxyribonucleic acid (DNA), ribonucleic acid (RNA), or DNA copies of ribonucleic acid (cDNA), all of which can be recombinantly produced, artificially synthesized, or isolated and purified from natural sources. The polynucleotides and nucleic acids may exist as single-stranded or double-stranded. The backbone of the polynucleotide can comprise sugars and phosphate groups, as may typically be found in RNA or DNA, or analogues or substituted sugar or phosphate groups. A polynucleotide may comprise naturally occurring or non-naturally occurring nucleotides, such as methylated nucleotides and nucleotide analogues (or analogs). The sequence of nucleotides may be interrupted by non-nucleotide components.

The term “promoter,” as used herein, generally refers to a nucleotide sequence, usually upstream (5′) to its coding sequence, which control the expression of the coding sequence by providing the recognition sequence for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or function RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene or be composed of different elements derived from different promoters found in nature, or even be comprised of synthetic DNA segments. A promoter may also contain DNA sequences that are involved in the binding of protein factors which control the efficacy of transcription initiation in response to physiological or developmental conditions.

“Inducible promoter,” as used herein, generally refers to those regulated promoters that can be turned on in a cell by an external stimulus, such as a chemical, light, hormone, stress, or a pathogen.

“Signal peptide,” as used herein, generally refers to those nucleic acid sequences that, when transcribed, result in the targeting of a functional protein or enzyme to a given locus within the cell, the cell membrane, or to the secretory pathway.

The term “enzyme,” as used herein, generally refers to a catalyst in various biological functions. For example, enzymes can help break down larger molecules of starch, fat, and protein during digestion by an organism. Enzymes can be proteins that help other organic molecules enter into chemical reactions with one another but are themselves unaffected by these reactions. In some cases, enzymes can comprise nucleic acids, such as RNA.

The term “protein,” as used herein generally refers to one or more nitrogenous organic compounds that comprise one or more chains of polypeptides. The term “polypeptides,” as used herein, generally refers to polymer chains comprised of amino acid residue monomers which are joined together through amide bonds (peptide bonds). The amino acids may be the l-optical isomer or the d-optical isomer. A polypeptide can be a chain of at least three amino acids, or a longer chain, e.g., at least 50, 100, 200 amino acids, or greater, in length. As used herein, a polypeptide may also comprise non-naturally occurring amino acids. As used herein, the abbreviations for the l-enantiomeric and d-enantiomeric amino acids that form a polypeptide are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). X or Xaa can indicate any amino acid.

The terms “designed” and “engineered,” as used herein, generally refer to polynucleotides, vectors, and nucleic acid constructs that have been genetically designed in silico and manipulated to encode a nutritional protein, an enzyme, a functional fragment of an enzyme, or another component of the animal feed described herein. An engineered polynucleotide, vector, or construct can be partially or fully synthesized in vitro. An engineered polynucleotide, vector, or construct can also be cloned. An engineered polyribonucleotide, vector, or construct can contain one or more base or sugar analogues, such as ribonucleotides not naturally-found in messenger RNAs. An engineered polyribonucleotide can contain nucleotide analogues that exist in transfer RNAs (tRNAs), ribosomal RNAs (rRNAs), guide RNAs (gRNAs), small nuclear RNA (snRNA), small nucleolar RNA (snoRNA), SmY RNA, spliced leader RNA (SL RNA), CRISPR RNA, long noncoding RNA (lncRNA), microRNA (miRNA), or another suitable RNA.

As used herein, “heterologous” or “exogenous” nucleic acid sequences or constructs or “transgenes” are generally DNA molecules that encode RNA and proteins that are not normally produced in vivo by the cell in which it is expressed. Any DNA that one of skill in the art would recognize or consider as heterologous or foreign to the cell in which it is expressed is herein encompassed by heterologous or exogenous DNA, or transgene. Examples of heterologous DNA include, but are not limited to, DNA that encodes enzymes, proteins, or another suitable component of animal feed on a bacterial cell, a fungus cell, an insect cell, an avian cell, a fish cell or a mammalian cell that normally does not express the molecule being encoded by the DNA.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5.

“Enrobing Agent” as used herein refers to a mixture of direct fed microbial in a mixture of water, oil, and prebiotic. The enrobing agent is applied directly to a complete animal feed pellet via spraying or pouring, and is mixed in industrial mixers for uniform application to the pellets.

The term “prebiotic” as used herein refers to any type of dietary fiber that acts as food for the microbes within the host microbiome, for example as a source of carbon.

### Transgenic Direct Fed Microbial Strains

In some instances, a nucleic acid construct, a vector, or an engineered fungus, bacteria, or yeast cell line of this disclosure comprises one or more nucleotide sequences that encode alpha-lactalbumin, ovalbumin, lactoferrin, lysozyme, lactoperoxidase, osteopontin, haptocorrin, alpha-amylase 1, bile-salt stimulated lipase, alpha-1-antitrypsin, myeloperoxidase, folate binding protein, insulin-like growth factor 1 (IGF-1), epidermal growth factor (EGF), orosomucoid, alpha-1-antichymotrypsin, alpha-1-b-glycoprotein, fetuin-A, alpha-enolase, alpha-S1-casein, kappa casein, beta-casein, alpha-s2-casein, caseinomacropeptide, copine-5, hapto-globin, hemoglobin subunit delta, lactadherin, CD14, mucin-1, mucin-16, mucin-4, serum albumin, serum transferrin, tenascin, thrombospondin-1, transthyretin, vitamin D-binding protein or vitronectin, a functional fragment of any one of the aforementioned proteins, or any combination of the enzymes, proteins, and functional fragments described herein. In some instances, the nucleic acid construct, vector, or composition also comprises the coding sequence of the avian, human, cow, or another mammalian protein encoding the molecules described above and the genetic code of 5′ untranslated regions (UTRs) and 3′ UTRs that facilitate expression in a yeast, fungal or in a bacterial cell.

In some embodiments, the expression levels of each protein range from 0.001% of total cellular protein to at least 30% of total cellular protein. In some embodiments, the expression levels of each protein range from 0.001% of total cellular protein to at least 40% of total cellular protein. In some embodiments, the expression levels of each protein range from 0.001% of total cellular protein to at least 50% of total cellular protein. In some embodiments, the expression levels of each protein range from 0.001% of total cellular protein to at least 75% of total cellular protein.

Intracellularly expressed proteins have the ability to significantly increase the intracellular presence of desired amino acids, e.g., essential amino acids, following expression of such proteins, by transporting amino acids into the cell from the peripheral growth medium. As a non-limiting example, lysine, which is a crucial amino acid in the growth and development of livestock, can be increased within a cell by expressing a protein in which the amino acid composition of the protein has about 2% or greater lysine content. Expression of such a protein can thus have positive effects on the amino acid profile of the microbial cell. In some embodiments, expression of proteins having a high content, e.g., about 2% or greater, of a desired amino acid, such as lysine, can increase the level of the desired amino acid within the cell by at least 1%, at least 2%, at least 5%, or even higher, compared to a counterpart control cell of the same strain that is not engineered to overexpress a protein comprising high levels of the amino acid. The resulting changes allow for the use of less product to more effectively deliver higher levels of the desired amino acid, e.g., lysine. Other amino acids for which it may be desirable to increase intracellularly include but are not limited to, threonine, methionine, and tryptophan.

In some embodiments, the protein that is expressed in the transgenic microbial strain, e.g., a yeast strain such as Sachharomyces cerevisiae, is substantially identical to naturally occurring chicken ovalbumin, chicken lysozyme, chick ovotransferrin, bovine beta-casein, bovine kappa casein, alpha-lactalbumin, bovine lysozyme, or a bovine lactoferrin sequence. Illustrative naturally occurring sequences are available under the following accession numbers: chicken ovalbumin, accession number A0A2H4Y842; chicken lysozyme, accession number B8YK79; chicken ovotransferrin, accession number E1BQC2; bovine beta-casein , accession number P02666; bovine kappa-casein, accession number P02668; bovine alpha-lactalbumin, accession number P00711; bovine lysozyme, accession number P04421 and bovine lactoferrin accession number 124627.

In some embodiments, the protein is substantially identical to one of SEQ ID NOS:1 to 8. In some embodiments, the protein has at least 70% or at least 75% identity to one of SEQ ID NOS:1 to 8. In some embodiments, the protein has at least 80% or at least 85% identity to one of

SEQ ID NOS:1 to 8. In some embodiments, the protein has at least 90% or at least 95% identity to one of SEQ ID NOS:1 to 8. In some embodiments, e.g., when the protein is a secreted protein, protein that is expressed is has at least 70%, at least 75%, at least 80%, at least 90%, or at least 95% identity to the region of SEQ ID NO:2, SEQ ID NO:3, or SEQ ID NO:7 that lacks the signal sequence. In some embodiments, e.g., when the polypeptide is targeted to the membrane, the polypeptide that is expressed in the transgenic direct fed microbial strain may additionally comprise a membrane targeting sequence that target the protein to the membrane in the desired microbial strain.

The term “substantially identical,” used in the context of two polypeptides, refers to a sequence that has at least 50% sequence identity with a reference sequence. “Percent identity” can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence, e.g., across the length of any one of SEQ ID NOS:1 to 8, using the programs described herein, preferably BLASTP using standard parameters.

Percent identity with respect to amino acid sequences is defined herein as the percentage of amino acid residues in the candidate sequence that are identical with the known polypeptides, after aligning the sequences for maximum percent identity and introducing gaps, if necessary, to achieve the maximum percent homology. Identity at the nucleotide or amino acid sequence level may be determined using methods known in the art, including but not limited to BLAST (Basic Local Alignment Search Tool) analysis using the algorithms employed by programs such as the BLAST programs blastp, blastn, blastx, tblastn and tblastx (Altschul (1997), Nucleic Acids Res. 25, 3389-3402, and Karlin (1990), Proc. Natl. Acad. Sci. USA 87, 2264-2268), which are tailored for sequence similarity/identity searching.

Protein sequences that are substantially identical to a reference sequence include “conservatively modified variants.” One of skill will recognize that individual substitutions in a polypeptide sequence that alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Examples of amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R) and His (Histidine or H); an “aromatic or cyclic group” including Pro (Proline or P), Phe (Phenylalanine or F), Tyr (Tyrosine or Y) and Trp (Tryptophan or W); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T) and Cys (Cysteine or C). Within each group, subgroups can also be identified. For example, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His and Trp; and the “phenyl sub-group” comprising Phe and Tyr. In another further example, the aliphatic group can be sub-divided into sub-groups including: the “large aliphatic non-polar sub-group” comprising Val, Leu and Ile; the “aliphatic slightly-polar sub-group” comprising Met, Ser, Thr and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH2 can be maintained.

In some embodiments, e.g., in which the polypeptide that is expressed in the transgenic direct-fed microbial strain is expressed intracellularly, the polypeptide need not retain catalytic function.

A recombinant protein can be modified by one or more post-translational modifications (PTMs). In some cases, a recombinant protein of this disclosure has a post-translational modification pattern that is associated with translation in vivo from fungal cells, bacterial cells, insect cells, or mammalian cells. PTMs are widely employed by all living organisms to control the enzymatic activity, localization or stability of proteins on a much shorter time scale than the transcriptional control, and the enzymes translated in a fungal or in a bacterial cell can have a different post-translational modification pattern as compared to an identical or similar enzyme translated in mammals. Enzymes can be extensively post-translationally modified in a fungus or in a bacterium by, for example, glycosylation, methylation, phosphorylation, acetylation, or ubiquitylation. In some cases, an enzyme can be chemically modified, for example, when translated within a yeast, fungus, or a bacterium. In preferred embodiments of this invention, the recombinant proteins exhibit a post-translational modification pattern characteristic for yeast cell, in particular, Saccharomyces cerevisiae (S. cerevisiae).

A PTM can involve the addition of hydrophobic groups that can target the polypeptide for membrane localization, the addition of cofactors for increased enzymatic activity, or the addition of smaller chemical groups. The encoded functional enzymes can also be post-translationally modified to receive the addition of sugar molecules, other peptides or protein moieties. A PTM can, for instance, extend the half-life of an enzyme or protein.

In some cases, the encoded enzyme(s) can be post-translationally modified within a host cell to undergo other types of structural changes. For instance, the encoded enzyme can be partially proteolytically cleaved. The encoded polypeptide can be folded intracellularly. In some cases, the encoded polypeptide is folded in the presence of co-factors and molecular chaperones. A folded polypeptide can have a secondary structure and a tertiary structure. A folded polypeptide can associate with other folded peptides to form a quaternary structure. A folded-peptide can form a functional multi-subunit complex, such as an antibody molecule, which has a tetrameric quaternary structure.

In yet other cases, the encoded protein(s) or enzyme(s) can be post-translationally modified within a host cell to change the chemical nature of the encoded amino acids. For instance, the encoded enzyme(s) can undergo post-translational citrullination or deimination, the conversion of arginine to citrulline. The encoded enzyme(s) can undergo post-translation deamidation; the conversion of glutamine to glutamic acid or asparagine to aspartic acid. The encoded enzyme(s) can undergo eliminylation, the conversion of an alkene by beta-elimination of phosphothreonine and phosphoserine, or dehydration of threonine and serine, as well as by decarboxylation of cysteine. The encoded enzyme(s) can also undergo carbamylation, the conversion of lysine to homocitrulline. An encoded enzyme(s) can also undergo racemization, for example, racemization of proline by prolyl isomerase or racemization of serine by protein-serine epimerase. The protein or enzyme that is translated within a bacterial or fungal cell can be structurally different as compared to an enzyme that is translated within a human or another mammalian or avian or fish cell.

In some embodiments, a recombinant protein can be encoded by more than one combination of codons in the degenerate code. In some embodiments, nucleotides are replaced by taking note of the genetic code such that a codon is changed to a different codon that codes for the same amino acid residue. A recombinant protein, or enzyme, of the disclosure can comprise one or more mutations as compared to the sequence disclosed herein. A mutation can be engineered within the gene of the recombinant protein such that the encoded amino acid is modified to a polar, non-polar, basic or acidic amino acid. As used herein, the term “mutated” or “replaced by another nucleotide” means one or more nucleotides at a certain position in a nucleotide sequence, vector, or in a construct that can be expressed heterologously in a fungus, bacteria, or another type of host cell is replaced at that position by a nucleotide other than that which occurs in the unmutated or previously mutated sequence. That is, in some instances, specific modifications may be made in different nucleotides.

A recombinant protein or an enzyme of this disclosure can comprise a molecular tag. A molecular tag can facilitate purification of a recombinant protein from a crude expression system. A molecular tag can be, for example, a polyhistidine tag, a glutathione-S-transferase (GST) tag, a maltose binding protein (MBP) tag, or a chitin binding protein (CBP) tag. A molecular tag can be present, for example, in the amino-terminus or in the carboxy terminus of a recombinant hen lysozyme and/or ovotransferrin protein.

In some embodiments, the production of the recombinant protein can be altered by engineering the direct fed microbial strain, e.g. via over-expression of genes for folding chaperones, over-expression of genes for trafficking proteins, reduction of intracellular and extracellular proteolysis, modification of promoter and signal sequences, increase of plasmid copy numbers, increasing the recombinant expression cassette copy number, as well as modulating the cultivation conditions.

### Engineered Fungi

In some embodiments, this invention relates to an engineered fungus comprising an endogenous or heterologous nucleic acid sequence that encodes one or more copies of the molecule described above. Genetically engineered fungi of the present disclosure may be constructed using a wide variety of techniques. For example, sequences encoding functional proteins or enzymes may be introduced at particular loci of the genome of the host cell by synthesizing oligonucleotides containing a tag, flanked by restriction sites enabling ligation to fragments of the native sequence. Following ligation, the resulting reconstructed sequence encodes a derivative having the sequence encoding a protein or functional enzyme. The fungi or bacteria may also be genetically modified using any available technology.

The genetic information encoding the heterologous enzyme or protein can be encoded in a vector or a construct that is stably integrated into the genome of the fungus.

In some embodiments of this invention, the construct comprises one or more motifs that facilitated the expression of the heterologous enzyme or protein in a fungus of the Aspergillus or Saccharomyces genus, such as Aspergillus oryzae, or S. cerevisiae.

Non-limiting examples of DNA motifs that are described in this invention include a promoter, a regulatory binding region, a gene, an allele, an intron, an exon, a gene cluster, a region encoding an enzyme active site, a region encoding a protein binding site, a region encoding a protein allosteric site, a combinatorial signature sequence, an aptamer, and fragments or combinations of any of the foregoing. Non-limiting examples of fungal and bacterial regulatory sequences that can be used include: 1) the FAKS secretion signal, TEF or GPD promoter sequence, the TEF or GPD terminator sequence in fungus of the Saccharomyces genus.

In some aspects, disclosed herein are vectors and constructs encoding genetically engineered proteins or enzymes which are operably linked to suitable transcriptional or translational regulatory elements for expression within a fungus or bacterium. In some embodiments of this invention, suitable regulatory elements are derived from fungal, bacterial, viral, mammalian, insect, or plant genes. Selection of appropriate regulatory elements is dependent on the chosen fungus or bacterial cell and, in some embodiments, includes: a transcriptional promoter and enhancer or RNA polymerase binding sequence, and a ribosomal binding sequence, including a translation initiation signal.

In some aspects of this invention, a transgene is operably linked to and/or contains at least one regulatory sequence, such as a promoter, an enhancer, an intron, a termination sequence, or any combination thereof, and, optionally, to a second polynucleotide encoding a signal sequence, which directs the enzyme encoded by the first polynucleotide to a particular cellular location e.g., an extracellular location. Promoters can be constitutive promoters or inducible (conditional) promoters.

In some embodiments, a variety of techniques may be used to genetically engineer fungal or bacterial cells. For example, cloned or synthetically engineered nucleic acid sequences can be inserted, replaced, or removed to generate an engineered fungus or bacterium with standard cloning techniques or genome editing techniques, such as: a) Homologous Recombination, b) the CRISPR/Cas9 systems; c) TALENs; d) ZFNs; e) and engineered meganuclease homing endonucleases. In some instances, these same fungal or bacterial cells can be further engineered to “knock-out” gene expression from one or more loci.

In some embodiments, non-limiting examples of proteins and enzymes that can be expressed in an engineered fungus, such as A. oryzae or S. cerevisiae, or in another type of host cell include hen lysozyme and/or ovotransferrin.

In some embodiments, non-limiting examples of proteins and enzymes that can be expressed in an engineered fungus, such as S. cerevisiae, or in another type of host cell include membrane bound, intracellular, or secreted hen lysozyme and/or ovotransferrin.

In some aspects, the expression cassette may include in the 5′-3′ direction of transcription, a transcriptional and translational initiation region, the polynucleotide of the invention and a transcriptional and translational termination region functional in vivo and/or in vitro. The termination region may be native with the transcriptional initiation region, may be native with the polynucleotide, or may be derived from another source. The regulatory sequences may be located upstream (5′ non-coding sequences), within (intron), or downstream (3′ non-coding sequences) of a coding sequence, and influence the transcription, RNA processing or stability, and/or translation of the associated coding sequence. Regulatory sequences may include, but are not limited to, enhancers, promoters, repressor binding sites, translation leader sequences, introns, and polyadenylation signal sequences. They may include natural and synthetic sequences as well as sequences which may be a combination of synthetic and natural sequences.

In various aspects of this invention, the expression of the transgene encoding the recombinant protein can be driven by native S. cerevisiae promotors. In some embodiments, native S. cerevisiae promotors of the TEF1, ADH1, TPI1, HXT7, TDH3, ADC1, PGK, GAPDH, PHO5, Gal1/Gal10, SUC2, MFα1, or ADRIII genes can be used for expression of heterologous or endogenous proteins or enzymes.

In some instances, the selection of an appropriate expression vector will depend upon the host cells, e.g. S. cerevisiae. In some cases, an expression vector contains (1) eukaryotic DNA elements coding for a fungal origin of replication and an antifungal (or antibiotic in case of bacterial host cells) resistance gene to provide for the amplification and selection of the expression vector in a fungal host (e.g. S. cerevisiae); (2) DNA elements that control initiation of transcription such as a promoter; (3) DNA elements that control the processing of transcripts such as introns, transcription termination/polyadenylation sequence; and (4) a gene of interest that is operatively linked to the DNA elements to control transcription initiation. The expression vector used may be one capable of autonomously replicating in the above host or capable of integrating into the chromosome, originally containing a promoter at a site enabling transcription of the linked phytase gene. In some aspects, yeast or fungal expression vectors may comprise an origin of replication, a suitable promoter and enhancer, and also any necessary ribosome binding sites, polyadenylation site, splice donor and acceptor sites, transcriptional termination sequences, and 5′ flanking nontranscribed sequences.

The expression cassette, or a vector construct containing the expression cassette, may be inserted into a cell, e.g. a fungal or bacterial cell. The expression cassette or vector construct may be carried episomally or integrated into the genome of the cell, e.g., derivatives of SV40; bacterial plasmids; phage DNA; baculovirus; yeast plasmids; or vectors derived from combinations of plasmids and phage DNA, viral DNA such as vaccinia, adenovirus, fowl pox virus, and pseudorabies, for instance. Any vector may be suitable as long as it is replicable and viable in the host cell.

In some embodiments, the plasmids utilized herein for transformation of S. cerevisiae are S. cerevisiae/E. coli shuttle vectors, with varying selection markers, promotors, terminators, and origin of replications. In some cases, the plasmid or vector includes several promoters (e.g. GPD(TDH3), TEF1,CYC1, and ADH1 promoters) cloned upstream of a multicloning site to allow for rapid and easy expression of heterologous genes. The vector may also comprise URA3,TRP1,HIS3, and LEU2 auxotrophic selection markers along with 2μ and CEN/ARS origins of replication.

A variety of techniques are available and known to those skilled in the art for introduction of constructs into a cellular host. Transformation of host cells, e.g. fungal or bacterial cells, may be accomplished through use of polyethylene glycol, calcium chloride, viral infection, DEAE dextran, phage infection, electroporation and other methods known in the art. In some embodiments of this invention, transformation of fungi may be accomplished according to Fincham et al. (Microbiol. Rev. 53:1, 148-170, 1989). Introduction of the recombinant vector into yeasts can be accomplished by methods including electroporation, use of spheroplasts, lithium acetate, and the like. Any method capable of introducing DNA into animal cells can be used. For example, electroporation, calcium phosphate, transient transfection, transient transformation, lipofection may be used for transformation of host cells.

In order to improve the ability to identify transformed host cells, one may desire to employ a selectable or screenable marker gene as, or in addition to, the expressible gene of interest. Furthermore, toxicity genes, auxotrophy genes, defective auxotrophy, and essential genes in the glycolytic pathway are commonly used as selective markers. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, i.e., through the use of a selective agent (e.g., an antibiotic, or the like), or whether it is simply a trait that one can identify through observation or testing, i.e., by ‘screening’. Of course, many examples of suitable marker genes are known to the art and can be employed in the practice of the invention. In some cases, more than one selection marker can be used to select a positive strain. For example, positive aLA mutants can be selected for using KanR and BleR selection markers.

In some aspects of this invention, the URA3 gene is utilized as a “marker gene” to label chromosomes or plasmids. URA3is a gene on chromosome V of S. cerevisiae and encodes for orotidine 5′-phosphate decarboxylase, which is an enzyme involved in the synthesis of pyrimidine ribonucleotides that allows for the selection of strains carrying the “marker gene” upon transformation.

In some cases, disclosed herein are nucleic acid sequences or constructs for transforming fungi of the Saccharomyces genus. In some cases, the construct can comprise one or more motifs that facilitated the expression of the heterologous enzyme or protein in the fungus. In general, such nucleic acids or constructs provide nucleic acid sequences that encode functional enzymes.

In some embodiments, methods for transforming or transfecting such fungal or bacterial cells to express exogenous enzymes are known in the art (see, e.g., Itakura et al., U.S. Pat. No. 4,704,362; Hinnen et al., PNAS USA 75, 1929-1933, 1978; Murray et al., U.S. Pat. No. 4,801,542; Upshall et al., U.S. Pat. No. 4,935,349; Hagen et al., U.S. Pat. No. 4,784,950; Axel et al., U.S. Pat. No. 4,399,216; Goeddel et al., U.S. Pat. No. 4,766,075; and Sambrook et al. Molecular Cloning. A Laboratory Manual, 2nd edition, Cold Spring Harbor Laboratory Press, 1989; for plant cells see Czako and Marton, Plant Physiol. 104, 1067-1071, 1994; and Paszkowski et al., Biotech. 24, 387-392, 1992). Representative methods include calcium phosphate mediated transfection, electroporation, lipofection, retroviral, adenoviral and protoplast fusion-mediated.

In some embodiments, the expression of a protein or enzyme in a fungus, for example lysozyme or ovotransferrin in S. cerevisiae, is optimized using a gene knock-in construct. In some instances, the T7 RNA polymerase gene is inserted into the chromosome along with the construct. The RNA polymerase can be placed immediately downstream of the xylA/xylR promoter/regulator. In some cases, the fungus strain can produce T7 RNAP upon induction, for example, upon induction with xylose. Selection markers can be used to select for positive fungus or bacterial strains expressing the construct.

In some embodiments, plasmid copy numbers and the mRNA level of the recombinant protein can depend and be altered by selecting a different marker type and/or promoter strength of the expression systems.

In some embodiments, a nucleic acid sequence or construct encoding alpha-lactalbumin can be inserted into the genome of fungus or bacteria for the generation of a stable mutant strain that is capable of producing high levels of heterologous protein. In some instances, bacterial alpha-Lactalbumin transcription can be controlled by the T7 promoter and terminated by the T7 Terminator sequence. Genetic engineering, such as insertions of heterologous DNA into the fungus or bacterium can be performed using homologous recombination, CRISPR/Cas9 technology, or other suitable methods.

Saccharomyces cerevisiae

Saccharomyces (S.) cerevisiae is a genetically well-known and well-characterized yeast species. Generally, yeasts, including S. cerevisiae are generally recognized as safe (GRAS) organisms by the FDA. The genome of S. cerevisiae comprises about 6000 genes. Furthermore, S. cerevisiae is capable of secreting proteins, expressing proteins on the cells surface, expressing proteins intracellularly, performing post-translational modification, and tolerates low pH, high sugar and ethanol concentrations and high osmotic pressure.

In some embodiments, the strains selected were the well-studied BY4741, BY4742, InvSc1, or a wild type strain isolated from Oak Trees in North Central California.

In some embodiments of this invention, promoters that initiate strong and constitutive expression are selected for recombinant protein production. In some instances, the widely used TEF1 promoter of S. cerevisiae can drive high gene expression in both high glucose conditions and glucose limited conditions. In some aspects, the TPI1 promoter of the strongly expressed glycolytic gene TPI1 of S. Cerevisiae may be used for production of recombinant proteins.

Different expression systems including the yeast integrative plasmids (YIps) or the yeast episomal plasmids (YEps), or homologously recombined de novo cassettes can be used in S. cerevisiae for integration of the desired gene into the yeast genome and/or for high copy number expression. In some aspects, the expression systems utilized in S. cerevisiae can harbor either single or multiple different promoters with varying regulation profiles and strengths, for instance. In various aspects of this invention, the bidirectional GAL1/GAL10 promoter cassette can be used in S. cerevisiae that provides the possibility of expressing two different genes at the same time from a single vector.

In some embodiments, yeast strains are propagated at 30° C. in YPD medium or yeast complete synthetic medium (CSM). YPD medium is composed of 10 g/L yeast extract, 20 g/L peptone, and 20 g/L glucose. CSM is composed of 6.7 g/L yeast nitrogen base, 20 g/L glucose, and either CSM, CSM-URA, CSM-HIS, CSM-LEU, or CSM-TRP additive (MP Biomedicals, Solon, Ohio), depending on the required auxotrophic selection.

### Application of Direct Fed Microbials to Animal Feed

To better ensure delivery of high quality, viable, direct fed microbial cells to the internal gut microbiome, also provided herein is an enrobing process for delivering the direct fed microbial to an animal.

An enrobing process of the present disclosure utilizes an oil and water emulsion in which the content of oil is from about 0.01% to about 10% of the total amount of feed weight. Water is added in a range of about 0.001% to about 10% of the total weight of the feed. Probiotics are added to the emulsion at about 0.01% to about 30% of the total feed weight. Prebiotics added to the pellets following application of the emulsion are supplied from about 0.01% to about 50% of the total feed weight. The enrobing process includes emulsifying pre-weighed oil, water and probiotics for at least half an hour in an industrial mixer, pre-weighing prebiotics and pelleted feed, and combining all of the ingredients into a larger industrial mixer. All processes typically occur at ambient conditions or about room temperature, e.g., from about 15° C. to about 21° C., up to the a maximum temperature that does not kill cells, e.g., up to a maximum of 42° C., 45° C., 50° C., or 55° C., depending on the microbial strain. The emulsion can be sprayed on to feed as it is mixing, or applied directly to the feed. The emulsion is added first and mixed with the feed to coat in the probiotic emulsion. The prebiotics are added secondarily to the feed which provides a powder coat to the pellets that prevent clumping of the final product, and deliver nutritional fiber to the microbes in the gut of the host.

The present disclosure is related to compositions for animal feed. The animal feed composition of the present disclosure can be supplemented to the animal at a time before, after, or simultaneously with the diet. In some embodiments, the direct fed microbial additive of the present disclosure is supplemented to the animal simultaneously with the diet. The composition comprising the recombinant strains of this disclosure may be combined with other ingredients to result in animal feed compositions with particular functionality and advantages.

In some embodiments, recombinant direct fed microbial strains of yeast, bacteria, or fungus are used to generate an enrobing agent when combined with oil, water, and pre-biotic substances that, after pre-mixing, are subsequently applied to an animal feed via spraying and/or mixing in an industrial mixer. The resulting “enrobed” animal feed is air dried and delivered orally to the animal. The direct fed microbial strain is then recovered from the microbiome of backyard, small-scale, or commercial livestock.

In some embodiments of this disclosure, the administered amount of direct fed microbial is from about 0.001 to 1 g per gram of food.

In some cases, the administered amount of direct fed microbial supplement is from about 0.001 to 1 g per kg body weight. In some cases, the administered amount of direct fed microbial supplement is from about 0.5 to 2 g per kg body weight. In some cases, the administered amount of direct fed microbial supplement is from about 1 to 5 g per kg body weight. In some cases, the administered amount of direct fed microbial supplement is from about 1 to 10 g per kg body weight. In some cases, the administered amount of direct fed microbial supplement is from about 1 to 50 g per kg body weight. In some cases, the administered amount of yeast supplement is from about 1 to 250 g per kg body weight. In some cases, the administered amount of yeast supplement is from about 1 to 500 g per kg body weight.

In some aspects, the present disclosure relates to a method for feeding an animal, comprising providing an animal feed comprising from about 1% to about 10% of a recombinant organism composition as described above, wherein the amounts are by total weight of the food, and providing the animal feed to the animal for ingestion. In some aspects, the animal feed comprises from about 0.001% to about 30% of a recombinant organism composition by total weight of the food. In some aspects, the animal feed comprises from about 1% to about 50% of a recombinant organism composition by total weight of the food. In some aspects, the animal feed comprises from about 1% to about 75% of a recombinant organism composition by total weight of the food.

### EXAMPLES

The following examples are offered to illustrate, but not to limit the claimed invention.

### Example 1. Nutritional Direct Fed Microbial Strain Construction and Validation

This example describes the construction of a nutritional yeast probiotic strain derived from parental strain PN002 (InvSc1) and its evaluation.

Non-secreted, intracellularly expressed proteins have the capability of altering the amino acid profile of any organism in which they are produced. To maximize the nutritional output of yeast, we co-expressed three bovine milk proteins and one hen egg protein intracellularly, within the yeast cell. These proteins were not purified, rather used to alter the amino acid profile of yeast. Nutritional proteins were chosen based on the presence of key amino acids within the protein that are otherwise limiting in yeast. This analysis was performed in silico with a protein analysis tool created in-house. Amino acids relevant to animal nutrition include: Tryptophan (Trp), Methionine (Met), Lysine (Lys), and Threonine (Thr). Bovine alpha-lactalbumin contains 3.2% Trp, 1.6% Met, 9.7% Lys, and 5.6% Thr. Bovine kappa-casein contains 0.6% Trp, 1.9% Met, 5.6% Lys, and 8.7% Thr. Bovine beta-casein contains 1.0% Trp, 3.3% Met, 5.2% Lys, and 4.3% Thr. Ovalbumin derived from the hen DNA sequence contains 1.0% Trp, 4.1% Met, 5.0% Lys, and 4.4% Thr. We hypothesized that by expressing these proteins simultaneously within the yeast cell, we would substantially increase the key amino acids of yeast, ultimately changing the amino acid profile of the organism. We also hypothesized that the resulting strain of yeast expressing these four proteins would better bind fat due to the fat-binding nature of the chosen proteins (e.g. kappa-casein).

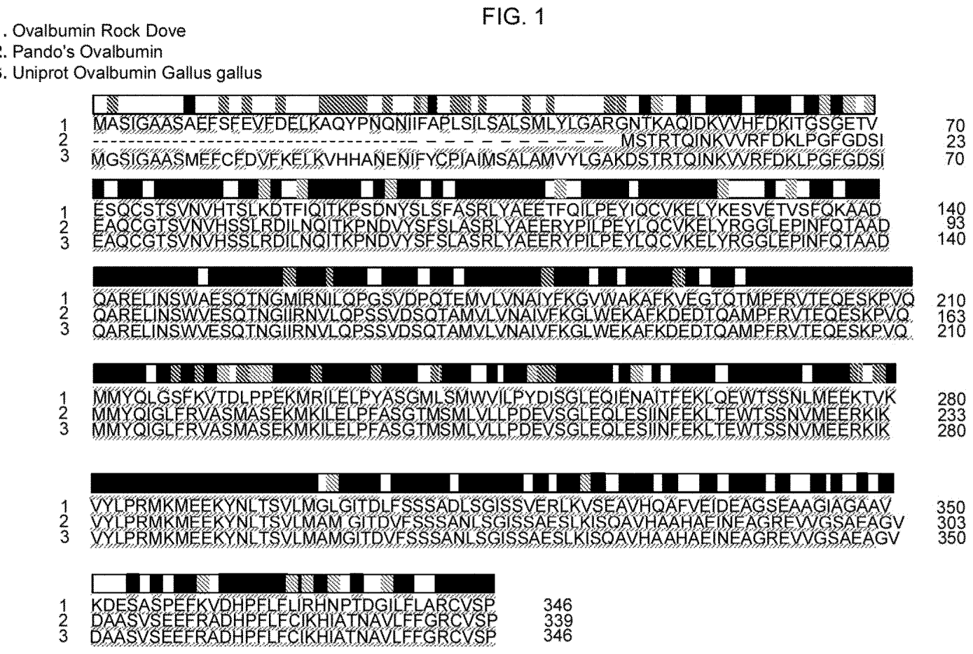

Each DNA sequence was designed in silico using Snapgene software, codon optimized for expression in S. cerevisiae, and edited in silico to exclude native signal sequences. The resulting protein sizes are as follows: bovine alpha-lactalbumin (14.3 kDa); bovine kappa-casein (17.9 kDa), bovine beta-casein (23.7 kDa) and Ovalbumin (37.7 kDa). Ovalbumin in particular is unique because the protein signal sequence is internal. FIG. 1 depicts the amino acid alignment of native hen ovalbumin compared to Pando Nutrition's Ovalbumin. Removing the first 49 amino acids of native hen Ovalbumin allows for the production of PN012-3, a strain that expresses a truncated, methionine-rich Ovalbumin along with bovine alpha-lactalbumin, bovine beta-casein, and bovine kappa-casein.

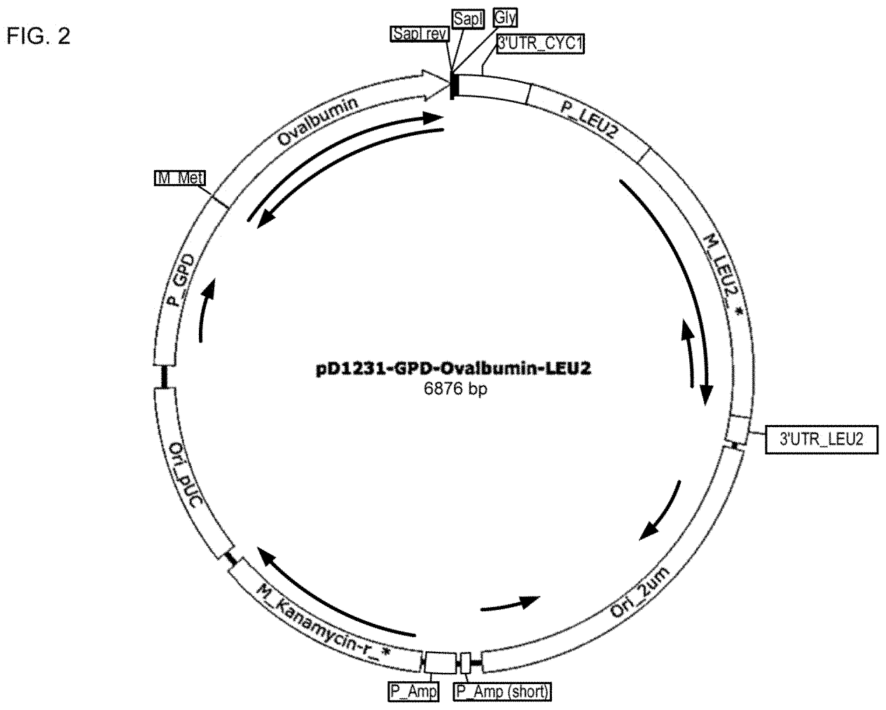

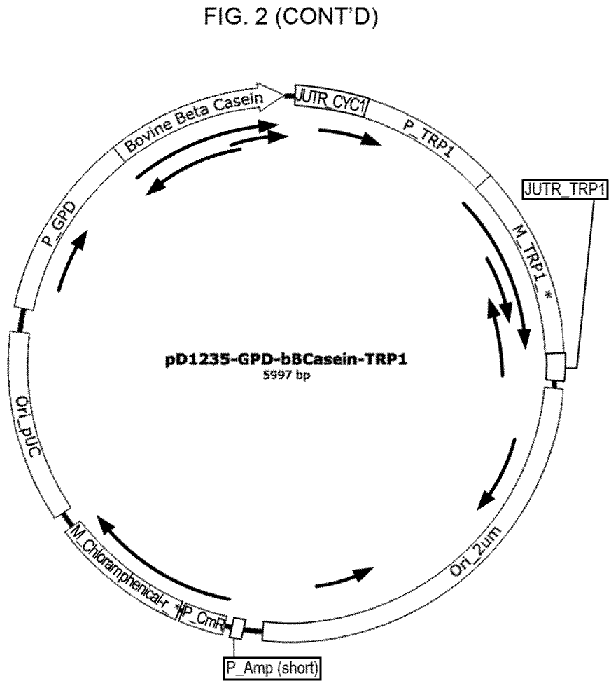

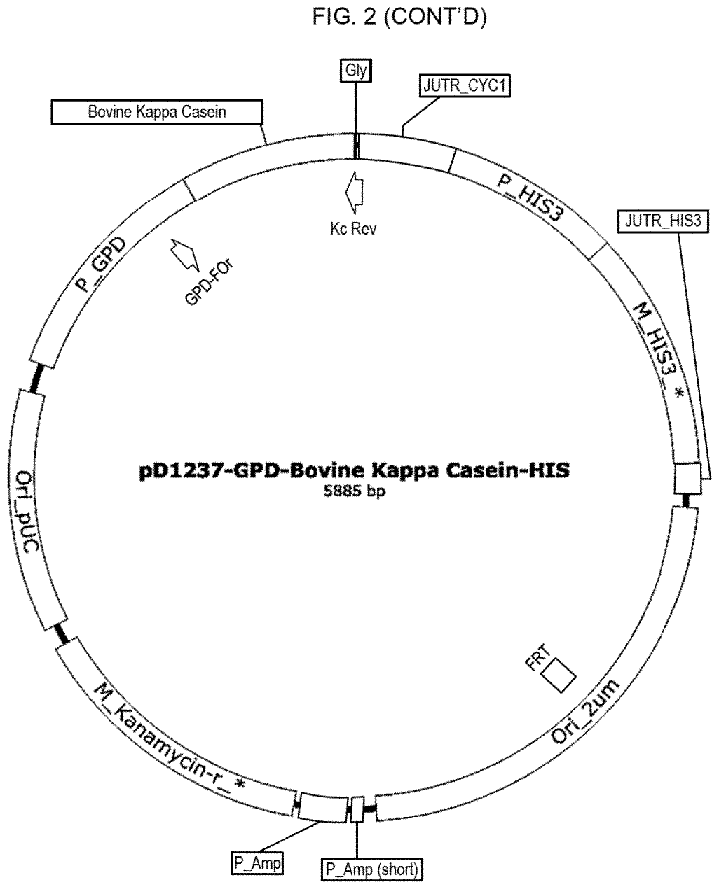

The designed DNA was synthesized by Integrated DNA Technologies (IDT, USA) and cloned directly into pD1231, pD1234, pD1235, and/or pD1237. These vectors were acquired from Atum Bio (USA) and lack a secretion signal. Using these plasmids allowed for the intracellular expression of our recombinant proteins. FIG. 2 depicts the plasmids created in this process. Each vector also contains an auxotrophic marker (LEU, URA, TRP, HIS) unique to each plasmid allowing for maintenance within the yeast host cell. The inserted genes included a 5′ and 3′ SapI site for directional cloning into the vectors. The cloned vectors were transformed into E. coli, purified, and Sanger Sequenced to determine proper insertion of the genetic cassette. The purified plasmids were transformed into yeast via chemical transformation (Lithium Acetate, Polyethylene glycol methods). The transformants were then selected on Complete Minimal Media (CMM: 10 g/L Glucose, 6.7 g/L Yeast Nitrogen Base, 2 g/L of synthetic dropout media lacking Leucine, Uracil, Tryptophan, and Histidine, with or without 20 g/L of Bacto Agar).

Purified plasmids were transformed using two methods. In one embodiment, the plasmids were transformed step wise, screened for expression, and taken into the subsequent round of transformation. The other method employed a 4-plasmid single transformation wherein all four recombinant plasmids were mixed in equimolar concentrations and chemically transformed into chemically competent PN002.

The first time the 4-plasmid reaction was transformed, colonies grew pseudohyphae on traditional selective media, which suggested there were not enough nutrients in the media to sustain budding yeast-cell growth. It was therefore essential to design a media for selection in which we increased the amount of glucose and nitrogen (CMM+: 20 g/L Glucose, 28 g/L Yeast Nitrogen Base, 2 g/L of synthetic dropout media lacking Leucin, Uracil, Tryptophan, and Histidine, with or without 20 g/L Bacto Agar). Subsequent co-transformation of the four plasmids and plating onto CMM+ media resulted in budding yeast colonies.

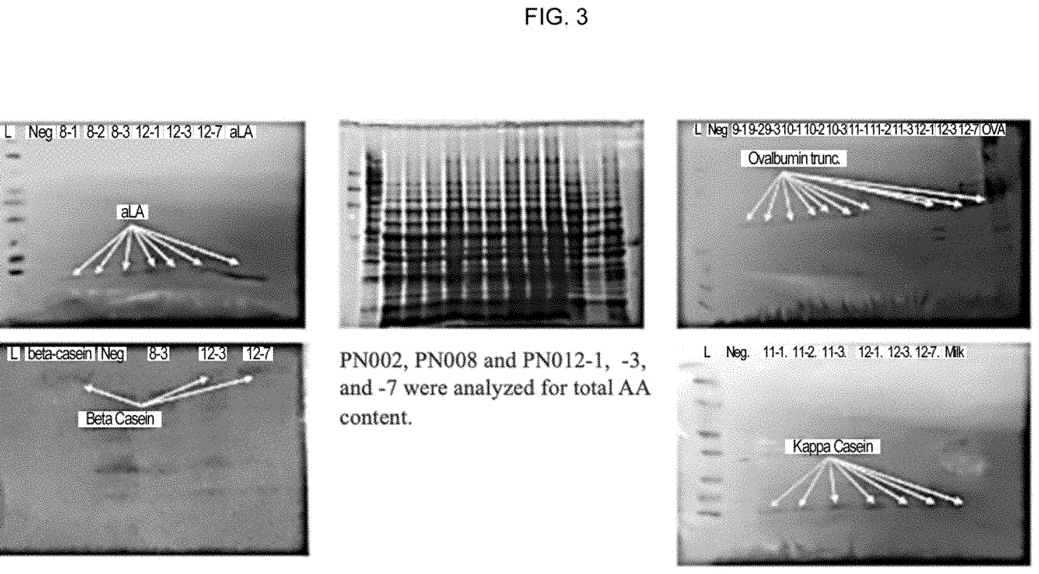

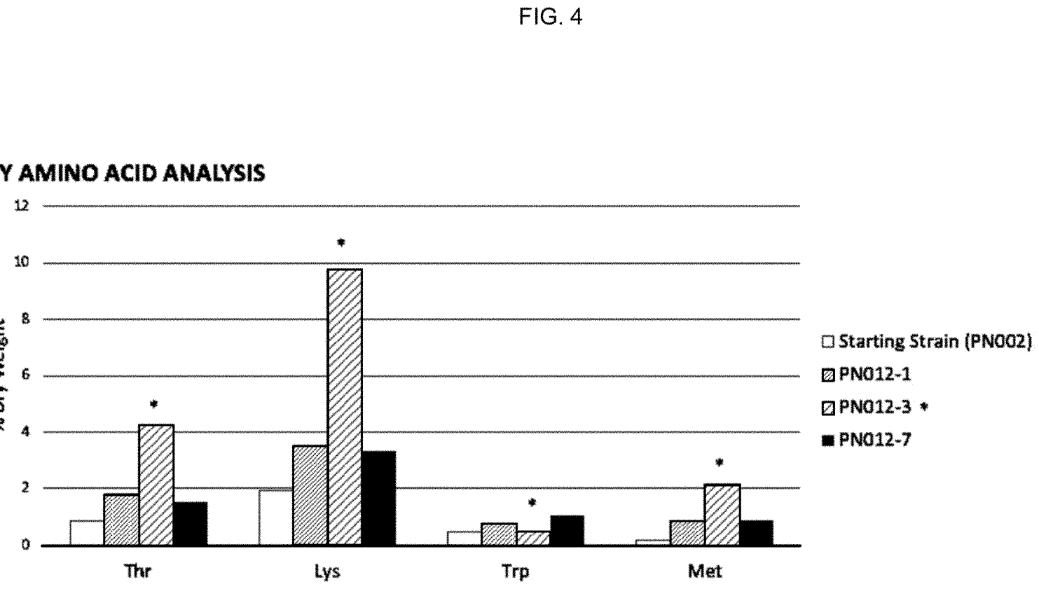

Following transformation of parental strain PN002, several colonies were screened for the presence of genes of interest via Western Analysis (FIG. 3). The results indicate that all proteins were expressed within the PN012-series of strains. Following growth in CMM+ broth media (CMM+ lacking bacto agar), cells were lysed with NaOH and boiling to release intracellular proteins. The lysates were analyzed by both SDS-PAGE and by transfer and detection on Nitrocellulose. Positive strains (PN012-1, PN012-3, PN012-7) were scaled in CMM+ Broth media and processed for amino acid analysis. FIG. 4 details the results of the amino acid analysis experiment. The results indicate that PN012-series of strains expressing four recombinant proteins contained higher levels of key amino acids transported into the cell via the growth medium when compared to their non-transformed counterpart (PN002). As shown in the figure, expression of these four proteins does in fact alter the amino acid profile of yeast, making it a more nutritious strain of yeast to be used in animal feed. Lysine, being a crucial amino acid in livestock growth, was used to determine the best clone. While the levels of lysine were increased in all strains, the greatest increase was observed in PN012-3. We therefore proceeded with this strain.

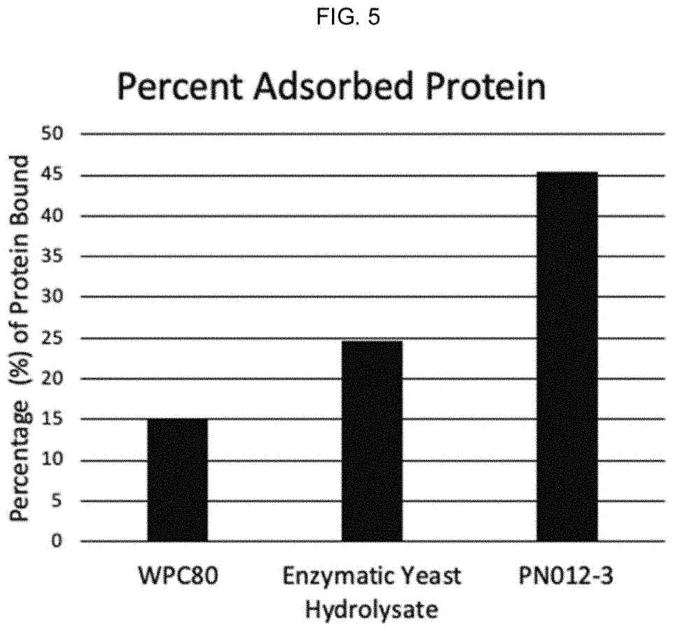

Fat is a crucial element of animal feed and methods to better deliver fat to livestock are needed in the animal feed space. The resultant strain in this example, PN012-3 was therefore tested for its ability to bind fat. We tested PN012-3 for its ability to adsorb fat when compared to other high-quality protein foods used in livestock. Briefly, PN012-3 was chemically disrupted, and emulsified in oil. The emulsion was centrifuged to pellet the micelles formed by the proteins in emulsion. The remaining protein in solution was quantified via a Bradford Protein Assay. The percent of protein remaining in the supernatant was subtracted from the total protein and divided by the total protein in solution prior to emulsification. The result was then multiplied by 100% to give the total of bound protein in solution. FIG. 5 details the results of this analysis and results indicate that more of the protein derived from PN012-3 is bound than protein from Enzymatic yeast hydrolysate or whey protein concentrate 80%. The ability of a direct fed microbial to bind fat when lysed has relevance in the field of animal nutrition. This ability has implications in the delivery of essential fatty acids to the host.

### Example 2. Transgenic, Transmembrane Direct Fed Microbial Strain Construction and Validation

This example describes one method of construction of a strain of S. cerevisiae PN002 expressing a transmembrane bovine lysozyme and its validation.

Lysozyme is a glycoside hydrolase that catalyzes the hydrolysis of 1,4-beta-linkages between acetylmuramic acid and N-acetyl-D-glucosamine residues in Peptidoglycan (PGC)—a component of the gram negative and positive bacterial cell wall. Expression of this protein by a direct fed microbial strain will result in the targeted destruction of cell wall components, and altered diversity of the gut microbiome.

The genetic insert was designed in silico to include a yeast-derived, transmembrane signal allowing for the addition of a Glycosylphsphatidylinositol (GPI) anchor to the C-terminus of newly expressed proteins. Immediately downstream of the signal peptide is the codon-optimized, bovine lysozyme gene, followed by a G₄S linker. Immediately downstream of the linker is the GPI anchor domain to which the GPI anchor is added in vivo by via processing through the endoplasmic reticulum. The transmembrane signal sequence was selected from the yeast genome based on known GPI-targeting proteins. Specifically, the signal peptide from the Gpi8p protein—a protein that adds transmembrane, GPI anchors to newly expressed proteins—was chosen based on its known localization within the yeast cell. The G₄S linker allows for flexibility in folding of the heterologous protein, and a space between the GPI anchor and the functional, bioactive protein. The GPI anchor motif allows for the addition of the GPI anchor, targeting to the membrane, and anchoring within the membrane of the yeast cell.

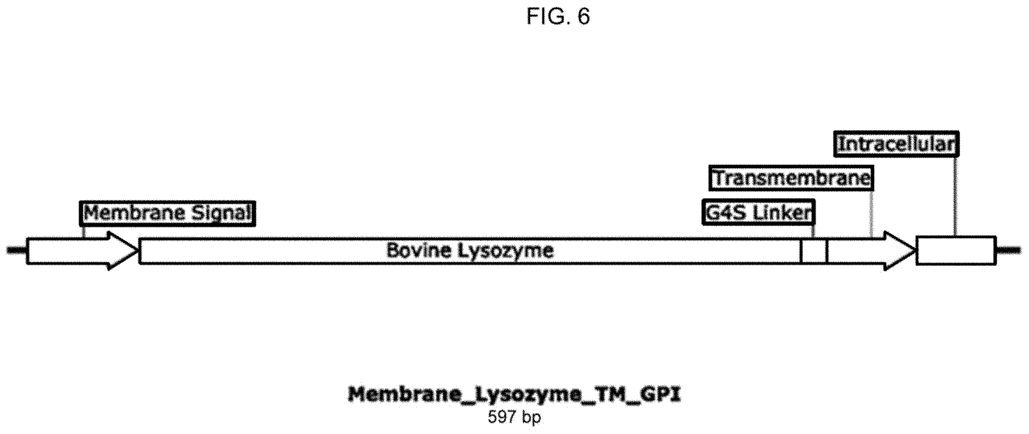

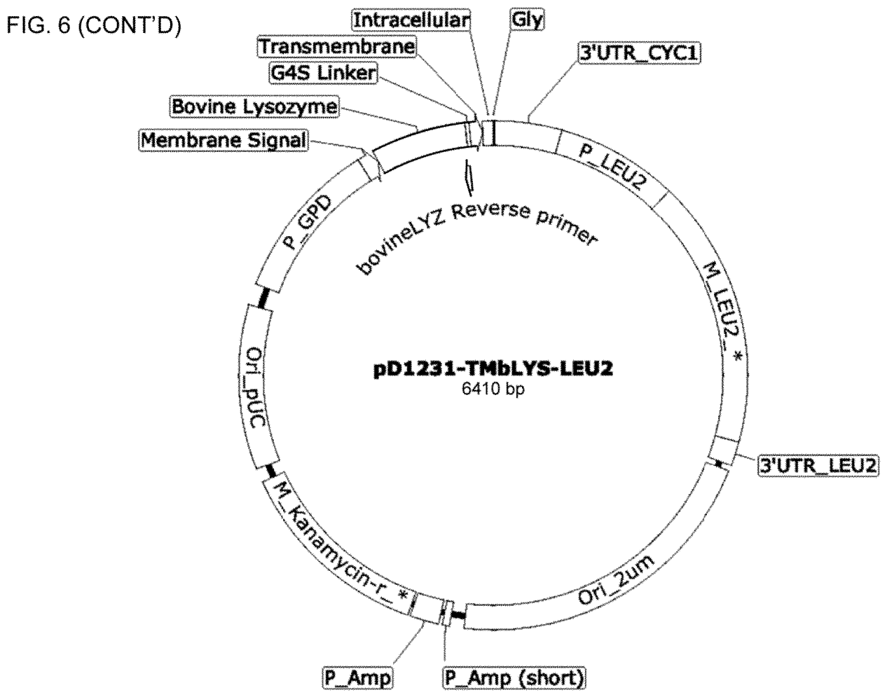

The transmembrane cassette was expressed in pD1231, cloned in frame using the SapI restriction sites. The resulting plasmid was transformed into Escherichia coli for propagation, purified, and sequence confirmed through traditional Sanger Sequencing methods. The sequence-confirmed plasmid was transformed into S. cerevisiae strain InvSc1 for expression analysis. The functional gene was synthesized by Genewiz, USA. FIG. 6 depicts the cassettes, plasmid backbone, and complete, sequence-confirmed plasmid used in the transformation of S. cerevisiae. Also indicated are the transmembrane signal and anchors that allow for proper insertion of the recombinant bovine lysozyme into the cell wall of PN002. The amino acid sequences of the transmembrane signal and GPI anchors that target protein to the membrane and allow for functional expression of full-length bovine lysozyme (PN030) or mutated bovine lysozyme (A114P, PN024) on the surface of yeast are:

Gpi8p Tm Signal-MRIAMHLPLLLLYIFLLPLSGA;
G4S spacer-GGGS
Gpi8p Transmembrane Domain-FKQSATIILALIVTILWFML
Gpi8p Intracellular Anchor-RGNTAKATYDLYTN

Two constructs were made during the course of this study. Briefly, PN024 contained the same cassette as pictured in FIG. 6 with a confirmed mutation at Amino Acid location 114 (A114P) from alanine to proline. The native construct was also expressed in PN031.

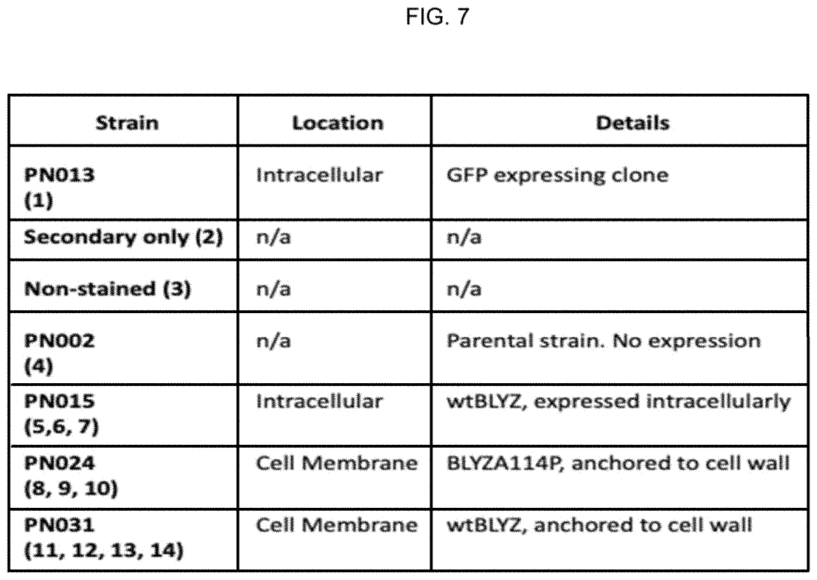

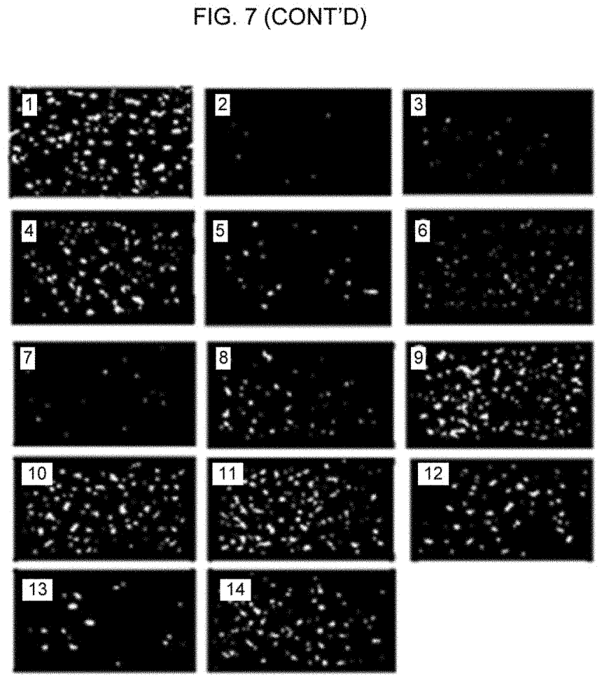

The resulting strain was confirmed for expression by immunofluorescence using a primary rabbit anti-bovine lysozyme antibody, and an anti-rabbit antibody linked to fluorescein isothiocyanate (FITC). The immunofluorescence data is depicted in FIG. 7. As shown in the figure, when compared to the non-transformed, parental strain, native bovine lysozyme (PN031) and mutated lysozyme (A114P, PN024) are both expressed on the surface of the yeast cell.

The resulting strains can either be grown for lysis and administration of yeast cell wall with lysozyme anchored into the membrane, or delivered as whole cell direct fed microbials. Data indicate that both forms of the protein delivery mechanisms result in functional lysozyme able to lyse Micrococcus luteus in lysis assays. The lysis assays were performed as follows. Briefly, yeast cells were either lysed or co-cultured with M. luteus in 1× Phosphate buffered saline. The results indicate that the bacteria are lysed

### Example 3. Bioactive-Direct Fed Microbial Strain Construction and Validation

This example describes the construction of a strain of S. cerevisiae expressing hen egg white lysozyme and/or ovotransferrin.

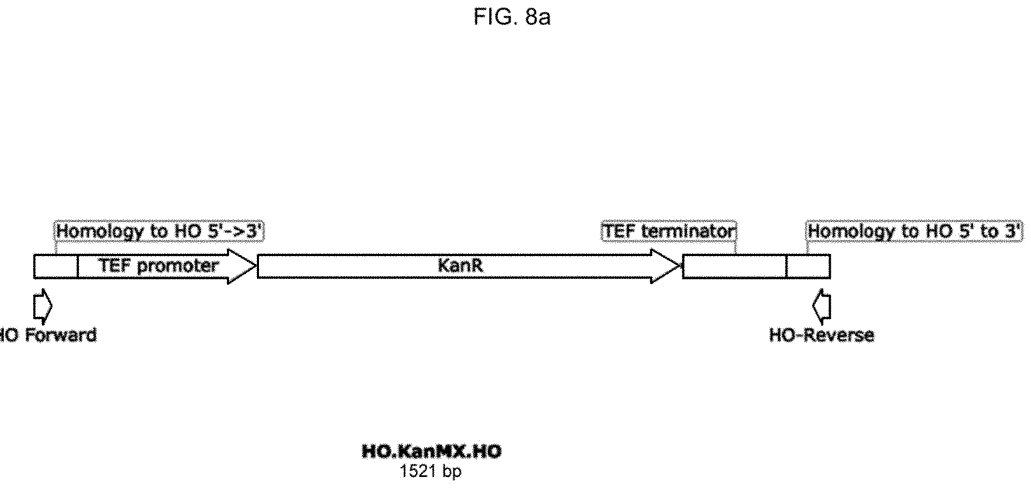

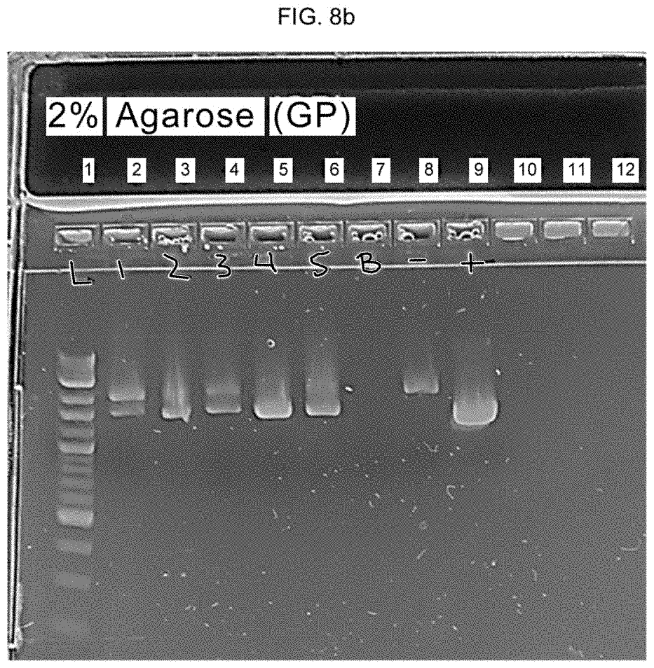

The knockout cassette targeting the wild-type yeast homothallism gene was synthesized by Genewiz, USA. The haploid BY4741 strain was altered by homologous recombination to remove the homothallism gene utilizing a KanMX cassette consisting of 5′ Homothallism gene homologies, the TEF promoter, Kanamycin Resistance Cassette, TEF Terminator, and 3′homothalism gene homologies. FIG. 8a depicts the knockout cassette.

The integration cassette was electroporated into wild type S. cerevisiae to knock out the homothallism gene, allowing for the selection of haploid spores of the MATa and MATα mating types. Following electroporation, the transformed cells were incubated on YPD+G418 (500 ug/mL) overnight. Positive colonies were screened by PCR for presence of the cassette. FIG. 8b depicts the amplicons from this reaction. Strains PN077-1, PN077-2, PN077-3, and PN077-5 were heterozygous for the KanMX allele. Strain PN077-4 was homozygous and was used in further experiments in which the chicken lysozyme cassette was knocked into the BY4741 strain. The heterozygous diploid strain PN077-4 was cultured overnight in G418 (500 ug/mL) and used as the starting strain for knocking in Lysozyme or Ovotransferrin.

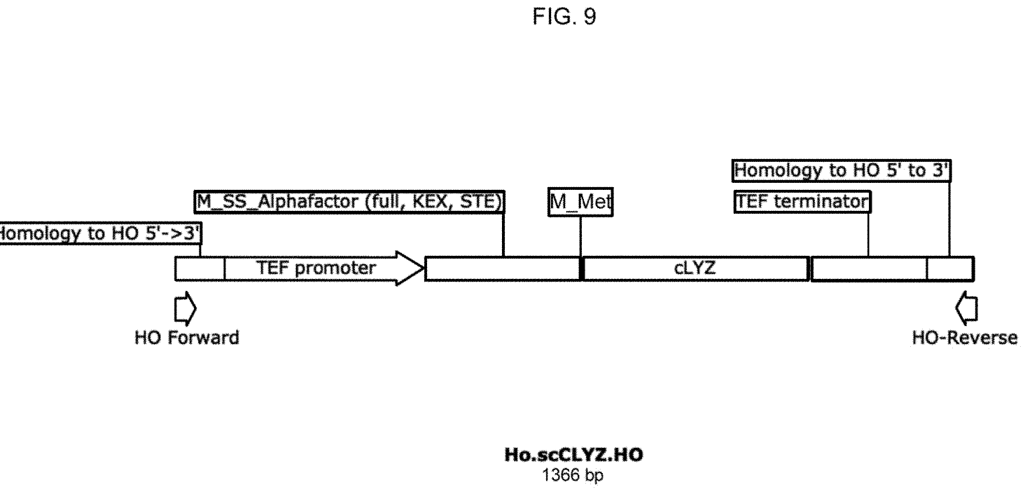

The ovotransferrin and hen egg lysozyme integration cassettes were codon optimized and constructed in silico. The cassettes were synthesized by Genewiz, USA. The cassette for secreted hen lysozyme is depicted in FIG. 9. Briefly, the TEF promoter is followed by the FAKS secretion signal, the codon optimized cassette and both 5′ and 3′ homothallism gene, homologous flanks. Following amplification and electroporation of integration cassettes expressing heterologous proteins, the haploid cells were plated onto YPD. Individual colonies were streaked onto YPD+G418 (500 ug/mL) and those that were sensitive to the antibiotic selection were screened by PCR to detect the presence of the Lysozyme or Ovotransferrin producing cassettes.

The positive haploid cells were then evaluated for their ability to produce Lysozyme in a Zone of Inhibition Assay. All clones (PN077-4-1 through -16 inhibited M. luteus in vitro.

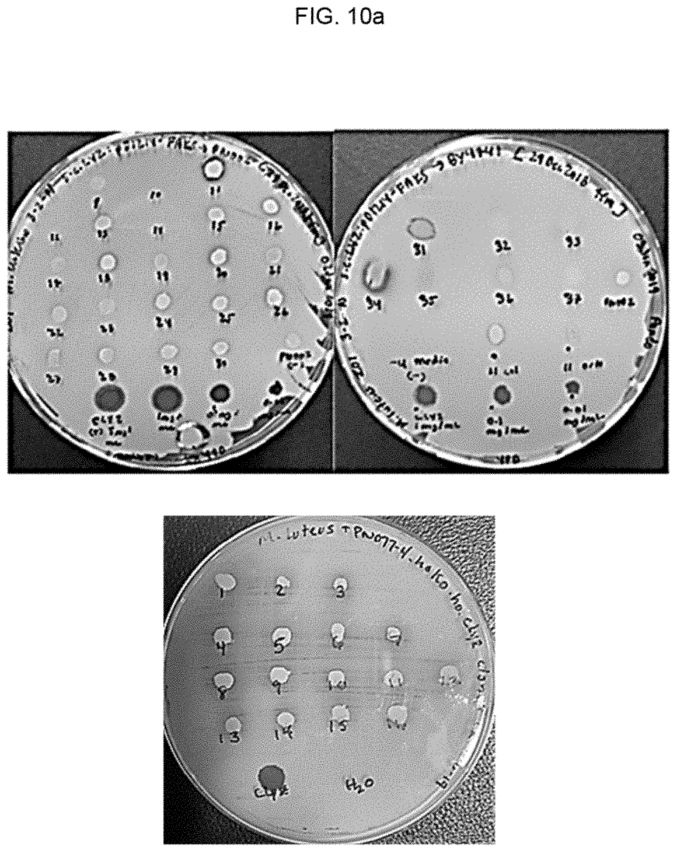

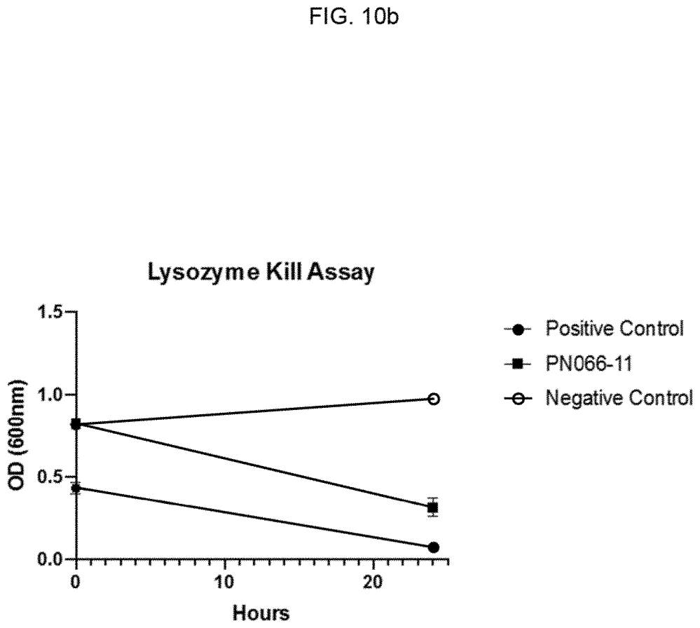

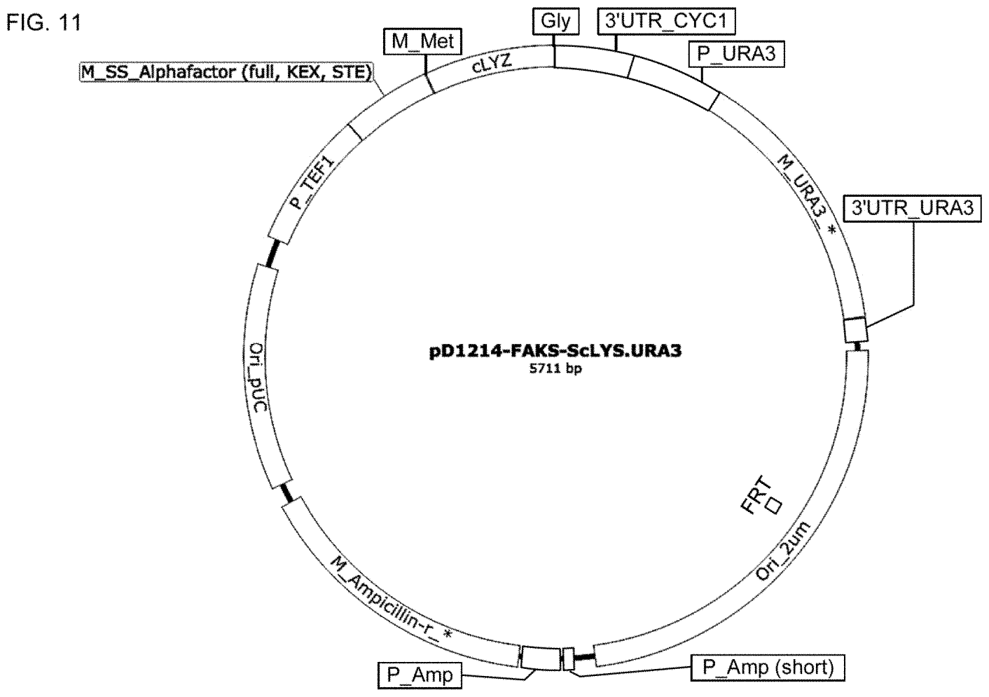

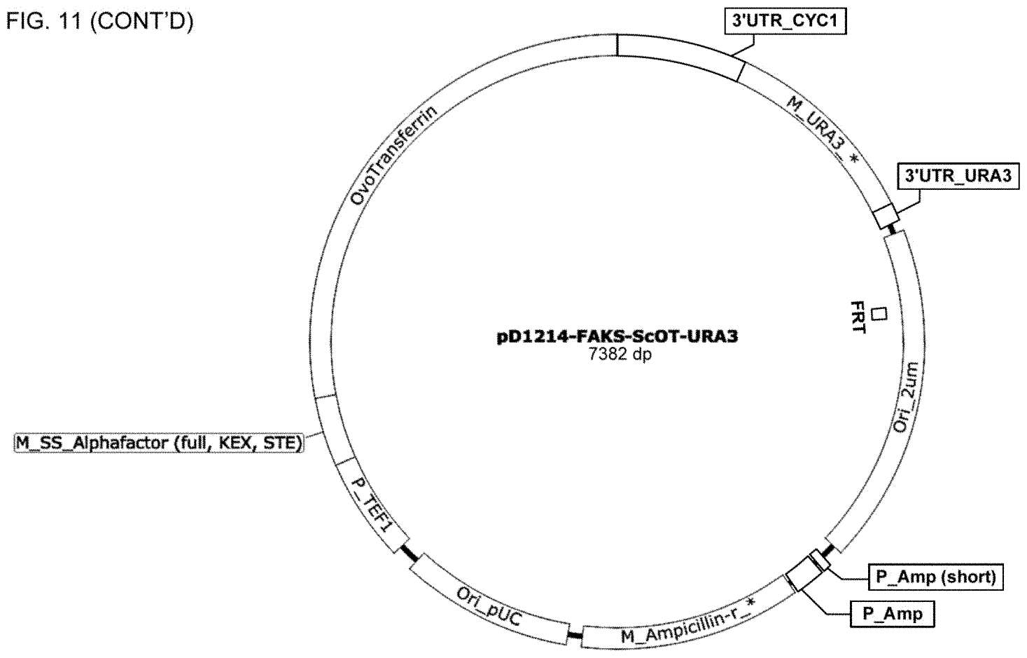

In some embodiments of this invention, plasmids pD1214-FAKS and pD1231 vectors (Atum Bio, USA) were used as backbones for the lysozyme or ovotransferrin genes. The resulting plasmids (FIG. 11) were transformed into BY4741 and PN002 and evaluated for efficacy via a zone of inhibition or kill assay using Micrococcus luteus as the target strain (FIGS. 10a and 10b). As seen in the figures, plasmid positive clones PN066-11 and PN067-31, and cassette integrated clones PN077-4-1 through -16 formed positive inhibitory rings when cultured with Microccocus luteus, which is an organism highly sensitive to lysozyme activity. Lysozyme prevents the growth of M. luteus, exhibited by a clearing around the strain secreting the enzyme.

### Example 4. Application of Transgenic Direct Fed Microbial Strains to Animal Feed

This example details the enrobing process used to deliver direct fed microbials to livestock and their recovery following the enrobing process in both in vitro and ex vivo conditions.

To determine the best method for enrobing feed with direct fed microbials, we evaluated several different methodologies. Beginning with the 10 g scale, we first weighed the feed pellets, and combined them with peanut oil at 1 g/10 g feed without water. This proved to be too oily for efficient coating. Also, without water, the direct fed microbial strain would not adhere to the feed.

The second method pre-mixed the direct fed microbial strain, prebiotics, and oil and attempted to apply the pre-mix to animal feed pellets. This mixture proved to be too viscous to effectively apply to the pellets.

The third attempt combined 0.5 g oil with 10 g of pellets, and added the prebiotic and direct fed microbial ingredients to the oil-coated pellets. This did not allow for efficient recovery of the direct fed microbial from the strain.

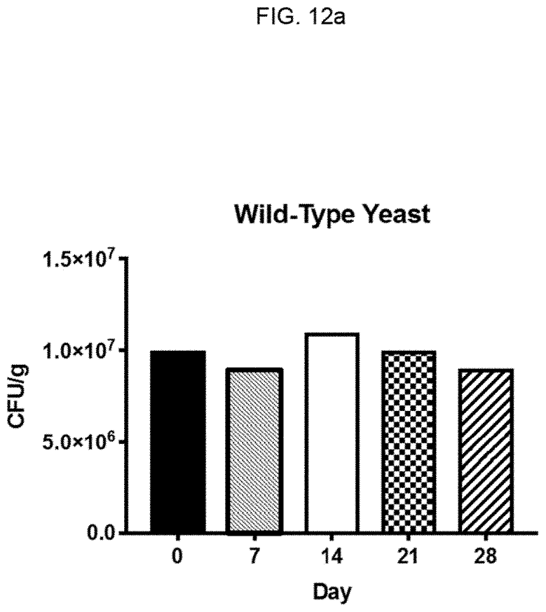

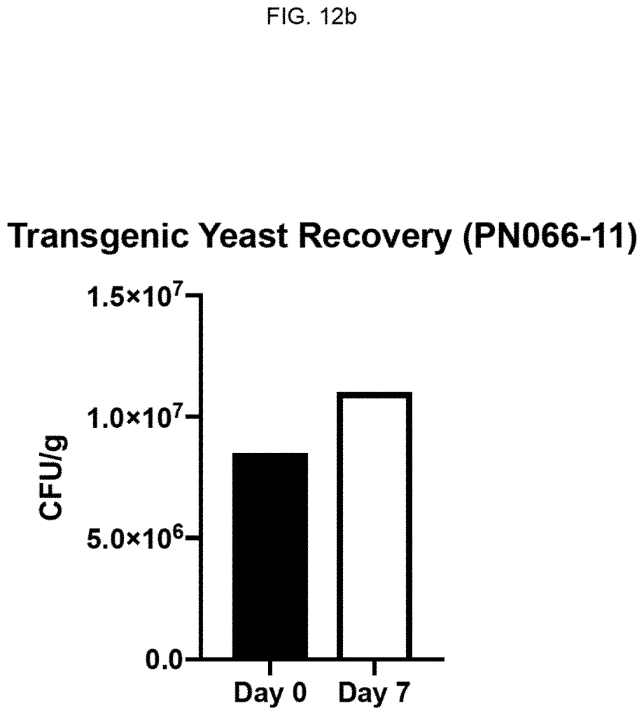

The fourth and final attempt premixed oil (0.5 g/10 g feed) with water 0.15% of total weight of feed and direct fed microbial at 0.01 g to 0.6 g per 10 g of feed. This pre-mix formed an emulsion after 30 minutes of agitation by vortex, kitchen mixer, or industrial mixer. The emulsion was applied to the feed pellets and mixed until an even coating was observed. Once the pellets were coated in emulsion, the pre-biotics were added to the pellets and mixed until the pellets were evenly coated (5-30 minutes). Following enrobing, the pellets were allowed to air dry 12 hours. Wild-type and recombinant direct fed microbial strains were then recovered in vitro at days 7, 14, and 28. FIGS. 12a and 12b depict their recovery. Wild type and recombinant, direct fed microbial strains of S. cerevisiae were successfully recovered in vitro following the enrobing process.

To test the efficiency of enrobing on delivering direct fed microbial strains, a beta-test of a commercial chicken treat product was performed in backyard layer hens. The treat was designed to deliver a full dose of direct fed microbial and prebiotics based on a 100 g diet by supplying the user with 10 g doses of treats. The study asked beta-testers to deliver 10 g of treat per chicken per day so that they would receive a full dose of direct fed microbial.

As an example, prebiotic Carrot Powder, Yeast Cell Wall, Wild-Type Yeast Strain PN030, water and Soybean Oil were used to coat chicken feed pellets. Carrot powder was chosen with the hope that the prebiotic would deliver a color change to the yolks of backyard chickens. Yeast cell wall was chosen because of its known effects on cholesterol in the eggs of backyard laying hens. Soybean oil was chosen based on its Omega-3 and Omega-6 composition, with the hope that it would confer changes in the Omega fatty acid profile of the egg yolks derived from backyard laying hens. Direct fed microbial strain PN030, a wild-type Oak strain of yeast, was chosen for its ability to grow to high densities in short periods of time.

For the beta test, one batch was equivalent to 25 lbs of final feed. For a 25 lb batch, 0.4 g of strain PN030 were mixed with 120 mL of distilled water and 5 lbs of soybean oil. These components were mixed in a kitchen aid mixer for 30 minutes, as the milky emulsion formed. While the emulsion was mixing, 25 lb of chicken feed pellets were weighed and added to an industrial mixer. Approximately 4 lb of carrot powder was weighed in a separate bowl and set aside. Yeast Cell Wall (0.25 lb) was also weighed and set aside. Following formulation of the emulsion, the industrial mixer containing pellets was started and the emulsion slowly added to the pellets. Once the pellets were evenly coated—as evidenced by a color change in the pellet—the pre-biotics were slowly added. Following addition of the prebiotics, the pellets were allowed to tumble in the industrial mixer for 5-30 minutes, until even distribution was observed. The pellets were then allowed to air dry overnight, and weighed out into 5 lb buckets for distribution to the beta-testers. Each bucket represented 28 days of a 10 g per chicken per day allotment.

Beta-testers (12 in total) were broken down into two groups: negative control (non-coated, chicken pellets without direct fed microbials) and experimental (direct fed microbial-coated pellets with prebiotics). Feed instructions were conveyed to the beta-testers, and the study lasted for 28 days. On days 0, 14, and 28 eggs were collected from the beta-testers and evaluated for yolk content and color. On days 0, and 28 fecal samples were collected from the flocks and analyzed by bacterial and fungal microbiome analysis.

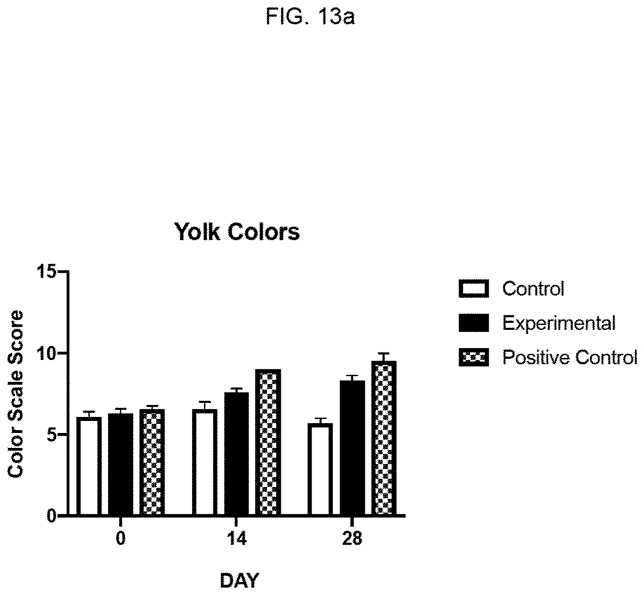

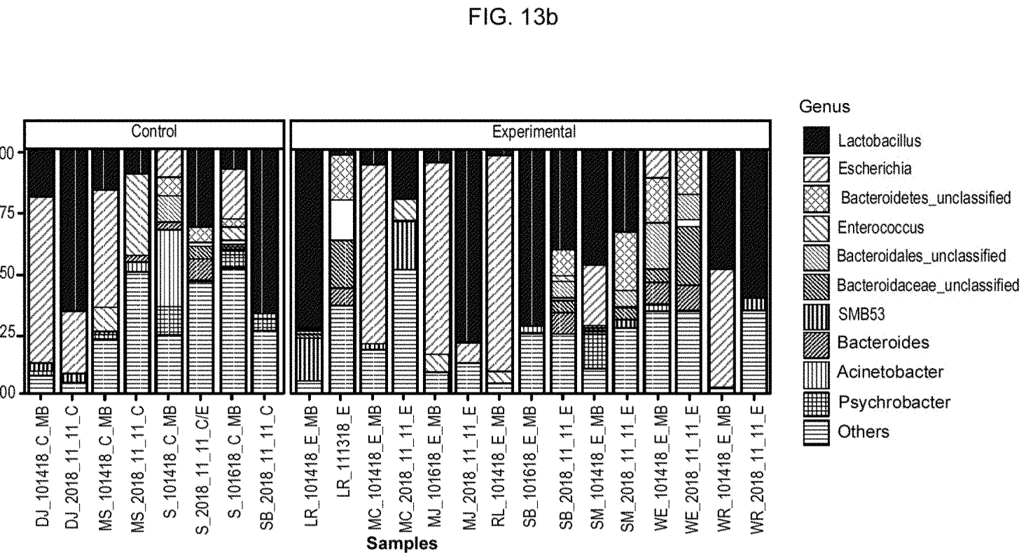

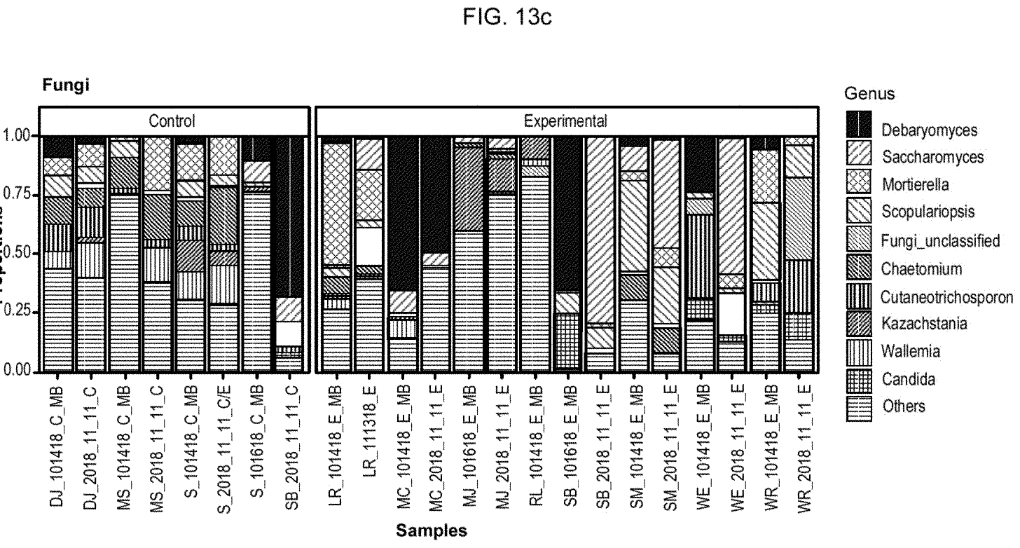

Results (FIG. 13a) indicate that the prebiotics were effectively delivered to the birds as evidenced by yolk color change. The data indicate that birds from experimental flocks laid eggs that had richer yolk colors, as evidenced by qualitative analysis. The microbiome was also positively affected, with a decrease in E. coli within the gut (FIG. 13b). Day 0 samples (“101418”) from experimentally treated birds indicated a presence of Escherichia species, that were eliminated by Day 28 (“2018_11_11”). Lactobacillus sp. were also increased in birds that received the direct fed microbial supplement. Yeast were also detected following the 28 day experiment, suggesting that our probiotic organism effectively alters the gut microbiome and is recoverable after a short period. The results indicate that samples at day 0 (“101418”) did not contain the allochthonous direct fed microbial, S. cerevisiae strain, while after 28 days of administration (“2018_11_11”) experimental groups did contain the yeast (FIG. 13c). Overall, these data demonstrate that the enrobing process is a cost- and time-effective way of delivering direct fed microbials to a livestock host.

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession number, and patent applications cited herein are hereby incorporated by reference in their entirety for all purposes.

### Illustrative Polypeptide Sequences:

```
SEQ ID NO: 1 chicken ovalbumin sequence:
                10 20 30 40
MGSIGAASME FCFDVFKELK VHHANETIFY CPIAIMSALA
                50 60 70 80
MVYLGAKDST RTQINKVVRF DKLPGFGDSI EAQCGTSVNV
                90 100 110 120
HSSLRDILNQ ITKPNDVYSF SLASRLYAEE RYPILPEYLQ
              130 140 150 160
CVKELYRGGL EPINFQTAAD QARELINSWV ESQTNGIIRN
              170 180 190 200
VLQPSSVDSQ TAMVLVNAIV FKGLWEKAFK DEDTQAMPFR
            210 220 230 240
VTEQEKPVQ MMYQIGLFRV ASMASEKMKI LELPFASGTM
              250 260 270 280
SMLVLLPDEV SGLEQLESII NFEKLTEWTS SNVMEERKIK
              290 300 310 320
VYLPRMKMEE KYNLTFVLMA MGITDVFSSS ANLSGISSAE
              330 340 350 360
SLKISQAVHA AHAEINEADR EVVGSAEAGV DAASVSEEFR
              370 380
ADHPFLFCIK HIATNAVLFF GRCVSP
SEQ ID NO: 2 chicken lysozyme:
                10 20 30 40
MRSLLILVLC FLPLAALGKV FGRCELAAAM KRHGLDNYRG
                50 60 70 80
YSLGNWVCAA KFESNFNTQA TNRNTDGSTD YGILQINSRW
                90 100 110 120
WCNDGRTPGS RNLCNIPCSA LLSSDITASV NCAKKIVSDG
              130 140
NGMNAWVAWR NRCKGTDVQA WIRGCRL
SEQ ID NO: 3 chicken ovotransferrin:
                10 20 30 40
MKLILCTVLS LGIAAVCFAA PPKSVIRWCT ISSPEEKKCN
                50 60 70 80
NLRDLTQQER ISLTCVQKAT YLDCIKAIAN NEADAISLDG
                90 100 110 120
GQAFEAGLAP YKLKPIAAEV YEHTEGSTTS YYAVAVVKKG
              130 140 150 160
TEFTVNDLQG KTSCHTGLGR SAGWNIPIGT LLHRGAIEWE
              170 180 190 200
GIESGSVEQA VAKFFSASCV PGATIEQKLC RQCKGDPKTK
              210 220 230 240
CARNAPYSGY SGAFHCLKDG KGDVAFVKHT TVNENAPDQK
              250 260 270 280
DEYELLCLDG SRQPVDNYKT CNWARVAAHA VVARDDNKVE
              290 300 310 320
DIWSFLSKAQ SDFGVDTKSD FHLFGPPGKK DPVLKDLLFK
              330 340 350 360
DSAIMLKRVP SLMDSQLYLG FEYYSAIQSM RKDQLTPSPR
              370 380 390 400
ENRIQWCAVG KDEKSKCDRW SVVSNGDVEC TVVDETKDCI
              410 420 430 440
IKIMKGEADA VALDGGLVYT AGVCGLVPVM AERYDDESQC
              450 460 470 480
SKTDERPASY FAVAVARKDS NVNWNNLKGK KSCHTAVGRT
              490 500 510 520
AGWVIPMGLI HNRTGTCNFD EYFSEGCAPG SPPNSRLCQL
              530 540 550 560
CQGSGGIPPE KCVASSHEKY FGYTGALRCL VEKGDVAFIQ
              570 580 590 600
HSTVEENTGG KNKADWAKNL QMDDFELLCT DGRRANVMDY
              610 620 630 640
RECNLAEVPT HAVVVRPEKA NKIRDLLERQ EKRFGVNGSE
              650 660 670 680
KSKFMMFESQ NKDLLFKDLT KCLFKVREGT TYKEFLGDKF
              690 700
YTVISSLKTC NPSDILQMCS FLEGK
SEQ ID NO: 4 bovine beta-casein:
                10 20 30 40
MKVLILACLV ALALARELEE LNVPGEIVES LSSSEESITR
                50 60 70 80
INKKIEKFQS EEQQQTEDEL QDKIHPFAQT QSLVYPFPGP
                90 100 110 120
IPNSLPQNIP PLTQTPVVVP PFLQPEVMGV SKVKEAMAPK
              130 140 150 160
HKEMPFPKYP VEPFTESQSL TLTDVENLHL PLPLLQSWMH
              170 180 190 200
QPHQPLPPTV MFPPQSVLSL SQSKVLPVPQ KAVPYPQRDM
              210 220
PIQAFLLYQE PVLGPVRGPF PIIV
SEQ ID NO: 5 bovine kappa-casein:
                10 20 30 40
MMKSFFLVVT ILALTLPFLG AQEQNQEQPI RCEKDERFFS
                50 60 70 80
DKIAKYIPIQ YVLSRYPSYG LNYYQQKPVA LINNQFLPYP
                90 100 110 120
YYAKPAAVRS PAQILQWQVL SNTVPAKSCQ AQPTTMARHP
              130 140 150 160
HPHLSFMAIP PKKNQDKTEI PTINTIASGE PTSTPTTEAV
              170 180 190
ESTVATLEDS PEVIESPPEI NTVQVTSTAV
SEQ ID NO: 6 bovine alpha-lactalbumin:
                10 20 30 40
MMSFVSLLLV GILFHATQAE QLTKCEVFRE LKDLKGYGGV
                50 60 70 80
SLPEWVCTTF HTSGYDTQAI VQNNDSTEYG LFQINNKIWC
                90 100 110 120
KDDQNPHSSN ICNISCDKFL DDDLTDDIMC VKKILDKVGI
              130 140
NYWLAHKALC SEKLDQWLCE KL
SEQ ID NO: 7 bovine lysozyme:
                10 20 30 40
MKALVILGFL FLSVAVQGKV FERCELARTL KKLGLDGYKG
                50 60 70 80
VSLANWLCLT KWESSYNTKA TNYNPSSEST DYGIFQINSK
                90 100 110 120
WWCNDGKTPN AVDGCHVSCR ELMENDIAKA VACAKHIVSE
              130 140
QGITAWVAWK SHCRDHDVSS YVEGCTL
SEQ ID NO: 8 bovine lactoferrin:
                10 20 30 40
MKLFVPALLS LGALGLCLAA PRKNVRWCTI SQPEWFKCRR
                50 60 70 80
WQWRMKKLGA PSITCVRRAF ALECIRAIAE KKADAVTLDG
                90 100 110 120
GMVFEAGRDP YKLRPVAAEI YGTKESPQTH YYAVAVVKKG
              130 140 150 160
SNFQLDQLQG RKSCHTGLGR SAGWIIPMGI LRPYLSWTES
              170 180 190 200
LEPLQGAVAK FFSASCVPCI DRQAYPNLCQ LCKGEGENQC
              210 220 230 240
ACSSREPYFG YSGAFKCLQD GAGDVAFVKE TTVFENLPEK
              250 260 270 280
ADRDQYELLC LNNSRAPVDA FKECHLAQVP SHAVVARSVD
              290 300 310 320
GKEDLIWKLL SKAQEKFGKN KSRSFQLFGS PPGQRDLLFK
            330 340 350 360
DSALGFLRIP SKVDSALYLG SRYLTTLKNL RETAEEVKAR
              370 380 390 400
YTRVVWCAVG PEEQKKCQQW SQQSGQNVTC ATASTTDDCI
              410 420 430 440
VLVLKGEADA LNLDGGYIYT AGKCGLVPVL AENRKSSKHS
              450 460 470 480
SLDCVLRPTE GYLAVAVVKK ANEGLTWNSL KDKKSCHTAV
              490 500 510 520
DRTAGWNIPM GLIVNQTGSC AFDEFFSQSC APGADPKSRL
              530 540 550 560
CALCAGDDQG LDKCVPNSKE KYYGYTGAFR CLAEDVGDVA
              570 580 590 600
FVKNDTVWEN TNGESTADWA KNLNREDFRL LCLDGTRKPV
              610 620 630 640
TEAQSCHLAV APNHAVVSRS DRAAHVKQVL LHQQALFGKN
              650 660 670 680
GKNCPDKFCL FKSETKNLLF NDNTECLAKL GGRPTYEEYL
              690 700
GTEYVTAIAN LKKCSTSPLL EACAFLTR
```

1. An animal feed supplement comprising a transgenic direct fed microbial strain genetically modified to express at least one polypeptide as follows:
an intracellularly expressed polypeptide selected from ovalbumin, bovine alpha-lactalbumin, bovine beta-casein, and bovine kappa casein, wherein the transgenic strain has an altered amino acid profile compared to a counterpart control of the same strain that does not have the genetic modification;
a membrane-anchored polypeptide selected from chicken lysozyme, bovine lysozyme, and chicken ovotransferrin; or
a secreted polypeptide selected from chicken lysozyme, bovine lysozyme and ovotransferrin.

2. The animal feed supplement of claim 1, wherein the transgenic direct fed microbial strain is genetically modified to express at least one intracellularly expressed polypeptide selected from ovalbumin, bovine alpha-lactalbumin, bovine beta-casein, and bovine kappa casein, and the transgenic strain has an altered amino acid profile compared to a counterpart control of the same strain that does not have the genetic modification.

3. The animal feed supplement of claim 2, wherein the animal feed supplement increases fat adsorption in the animal to which it is fed.

4. The animal feed supplement of claim 2, wherein the transgenic direct fed microbial strain is genetically modified to express an ovalbumin that lacks a secretion signal.

5. (canceled)

6. The animal feed supplement of claim 1, wherein the transgenic direct fed microbial strain expresses membrane-anchored chicken ovotransferrin, or membrane-anchored chicken or bovine lysozyme.

7. (canceled)

8. The animal feed supplement of claim 1, wherein the transgenic direct fed microbial strain expresses secreted bovine or chicken lysozyme.

9. The animal feed supplement of claim 1, wherein the transgenic direct fed microbial strain expresses secreted ovotransferrin.

10. The animal feed supplement of claim 1, where the transgenic direct fed microbial strain is a Saccharomyces cerevisiae genetically modified to express the at least one secreted polypeptide and the gene encoding the secreted polypeptide encodes the region of the polypeptide that is secreted fused to a yeast FAKS secretion signal.

11. The animal feed supplement of claim 1, wherein the microbial strain that is genetically modified to produce the transgenic direct fed microbial strain is an allochthonous yeast species.

12. The animal feed supplement of claim 1, wherein the transgenic direct fed microbial strain is a Saccharomyces cerevisiae.

13.-14. (canceled)

15. A method of formulating an animal feed comprising an animal feed supplement of claim 1 for feeding to animals, comprising enrobing the transgenic microbial strain, wherein the step of enrobing comprises resuspending the transgenic microbial strain in an emulsion of fat, water and prebiotics, and coating onto feed pellets.

16. (canceled)

17. An animal feed formulated by the method of claim 15.

18. A method of formulating an animal feed comprising an animal feed supplement of claim 2 for feeding to animals, comprising enrobing the microbial strain, wherein the step of enrobing comprises resuspending the transgenic microbial strain in an emulsion of fat, water and prebiotics, and coating onto feed pellets.

19. (canceled)

20. An animal feed formulated by the method of claim 18.

21. A method of increasing fat adsorption in an animal, the method comprising feeding the feed of claim 20 to an animal.

22. The method of claim 21, wherein the animal is a chicken.

23. A method of altering the content of gram positive bacteria in the microbiome of an animal, the method comprising feeding the animal feed supplement of claim 8 to an animal.

24. (canceled)

25. A method of altering the content of gram negative bacteria in the microbiome of an animal, the method comprising feeding the animal feed supplement of claim 9 to an animal.

26-27. (canceled)

28. An animal feed supplement comprising a transgenic direct fed microbial strain that is genetically modified to express a heterologous recombinant protein, or fragment thereof, selected from the group consisting of: an alpha-lactalbumin, an ovalbumin, a lactoferrin, a lysozyme, a lactoperoxidase, an osteopontin, a haptocorrin, an alpha-amylase 1, a bile-salt stimulated lipase, an alpha-1-antitrypsin, a myeloperoxidase, a folate binding protein, an insulin-like growth factor 1 (IGF-1), an epidermal growth factor (EGF), an orosomucoid, an alpha-1-antichymotrypsin, an alpha-1-b-glycoprotein, a fetuin-A, an alpha-enolase, an alpha-S1-casein, a kappa casein, a beta-casein, an alpha-s2-casein, a caseinomacropeptide, a rcopine-5, a hapto-globin, a hemoglobin subunit delta, a lactadherin, a CD14, a mucin-1, a mucin-16, a recombinant mucin-4, a serum albumin, a t serum transferrin, a tenascin, a thrombospondin-1, a transthyretin, a vitamin D-binding protein, and a vitronectin protein.

29. (canceled)

30. The animal feed supplement of claim 28, wherein the protein is present in the transgenic direct fed microbial strain in an amount between 5×10⁻¹⁵ g and 5×10⁻¹² g, or 0.1% to 100% of total cellular protein weight.

31. The animal feed supplement of claim 28, wherein the amino acid profile of the transgenic direct fed microbial strain is altered compared to a counterpart of the same strain that does not have the genetic modification.

32. The animal feed supplement of 28, wherein the transgenic direct fed microbial strain is a yeast strain, of the genus Aspergillus, or of the genus Bacillus.

33.-39. (canceled)
